(19) United States
(12) Patent Application Publication
(10) Pub. No.: US 2026/0335451 A1
(43) Pub. Date: Oct. 29, 2026

(54) SOLID ORAL DOSAGE FORMS OF ESTROGEN RECEPTOR DEGRADERS

(71) Applicant: Arvinas Operations, Inc., New Haven, CT (US)

(72) Inventors: Royal J. Haskell, III, Durham, CT (US); Joseph P. Reo, Riegelsville, PA (US)

(21) Appl. No.: 19/491,505

(22) PCT Filed: Jun. 11, 2024
(86) PCT No.: PCT/US2024/033417

§ 371 Date: Dec. 9, 2025

Related U.S. Application Data
(60) Provisional application No. 63/472,523, filed on Jun. 12, 2023.

(51) Int. Cl. A61K 47/55; A61K 9/16; A61K 9/20; A61K 47/54; A61K 47/69

(52) U.S. Cl. CPC A61K 47/55; A61K 9/1641; A61K 9/1652; A61K 9/1694; A61K 9/2009; A61K 9/2013; A61K 9/2018; A61K 9/2031; A61K 9/2054; A61K 47/545; A61K 47/6943

(57) ABSTRACT

Disclosed herein are solid oral dosage forms comprising Compound A, or a pharmaceutically acceptable salt thereof.

### RELATED APPLICATIONS

This application claims priority to, and the benefit of U.S. Provisional Application No. 63/472,523, filed Jun. 12, 2023, the contents of which are incorporated herein by reference in their entirety for all purposes.

### BACKGROUND

Certain bifunctional compounds target specific cellular proteins for degradation via the ubiquitin-proteasome system. Examples of such proteolysis targeting chimeric compounds (i.e., “PROTAC® protein degraders”) that target the estrogen receptor (ER) for ubiquitination and subsequent degradation are disclosed in International Publication No. WO 2018/102725, which is incorporated herein by reference in its entirety. Such bifunctional molecules exhibit a range of pharmacological activities consistent with the degradation of the ER including, but not limited to, treatment or amelioration of a disease condition such as cancer (e.g., breast cancer, uterine cancer, ovarian cancer, prostate cancer, endometrial cancer), or endometriosis.

A bifunctional molecule of particular interest is (S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione or (3S)-3-[1,3-dihydro-1-oxo-5-[4-[[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (referred to herein as “Compound A” or “Cpd A”), which has the molecular formula of C₄₅H₄₉N₅O₄ and the following structure:

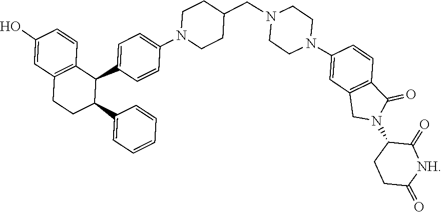 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is under development as a PROTAC® protein degrader that targets ER for the potential treatment of breast cancer and has been shown to be a useful modulator of targeted protein ubiquitination and degradation via the ubiquitin-proteasome pathway.

There is a need to develop dosage forms of Compound A, including those suitable for oral administration. Such dosage forms may have certain advantages (e.g., increased oral bioavailability) and may be useful for safe, effective and/or convenient administration of Compound A to patients for, e.g., treating cancers (e.g., breast cancer).

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Disclosed herein, in part, are solid oral dosage forms (e.g., tablets) comprising Compound A:

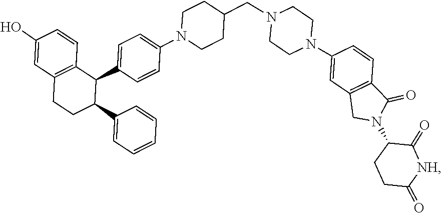 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

or a pharmaceutically acceptable salt thereof. Particularly, disclosed herein are solid oral dosage forms comprising Compound A, or a pharmaceutically acceptable salt thereof, a polymer, and a surfactant. In embodiments, the amount of Compound A in the solid oral dosage form is about 5 mg to about 500 mg. In embodiments, the solid oral dosage form is a tablet, a sachet, or a capsule.

In embodiments, the solid oral dosage form further comprises one or more excipients selected from fillers, disintegrants, glidants, and lubricants. Exemplary fillers include, but are not limited to, microcrystalline cellulose, silicified microcrystalline cellulose, lactose monohydrate, mannitol, sorbitol, xylitol, hydroxypropyl methylcellulose, hydroxypropyl cellulose, pullulan, fast-dissolving carbohydrates, and combinations thereof. Exemplary disintegrants include, but are not limited to, sodium starch glycolate, sodium carboxymethyl cellulose, calcium carboxymethyl cellulose, croscarmellose sodium, crospovidone, chitosan, agar, alginic acid, calcium alginate, methyl cellulose, microcrystalline cellulose, powdered cellulose, lower alkyl substituted hydroxypropyl cellulose, hydroxylpropyl starch, low-substituted hydroxypropylcellulose, polacrilin potassium, starch, pregelatinized starch, sodium alginate, polacrilin potassium, povidone, and combinations thereof. Exemplary glidants include, but are not limited to, silicon dioxide, colloidal silicon dioxide, calcium silicate, magnesium silicate, magnesium trisilicate, talc, starch, and combinations thereof. Exemplary lubricants include, but are not limited to, magnesium stearate, calcium stearate, glyceryl monostearate, glyceryl behenate, glyceryl palmitostearate, hexagonal boron nitride, hydrogenated vegetable oil, light mineral oil, mineral oil, polyethylene glycol, poloxamer, sodium benzoate, sodium lauryl sulfate, sodium stearyl fumarate, stearic acid, talc, zinc stearate, and combinations thereof.

In embodiments, the solid oral dosage form is a tablet. In embodiments, the tablet is film coated.

In embodiments, the solid oral dosage form (e.g., the tablet) comprises:
about 15% w/w to about 50% w/w of Compound A;
about 10% w/w to about 40% w/w of hydroxypropyl methylcellulose;
about 0.5% w/w to about 5% w/w of D-α-tocopheryl polyethylene glycol succinate;
about 10% w/w to about 40% w/w of microcrystalline cellulose;
about 5% w/w to about 15% w/w of lactose monohydrate;
about 5% w/w to about 15% w/w of croscarmellose sodium;
about 0% w/w to about 5% w/w of silicon dioxide; and
about 0% w/w to about 2% w/w of sodium stearyl fumarate.

In embodiments, the solid oral dosage form (e.g., the tablet) comprises:
15% to 50% w/w of Compound A;
10% to 40% w/w of hydroxypropyl methylcellulose;
0.5% to 5% w/w of D-α-tocopheryl polyethylene glycol succinate;
10% to 40% w/w of microcrystalline cellulose;
5% to 15% w/w of lactose monohydrate;
5% to 15% w/w of croscarmellose sodium;
0% to 5% w/w of silicon dioxide; and
0% to 2% w/w of sodium stearyl fumarate.

In embodiments, the solid oral dosage form (e.g., the tablet) comprises:
about 42% w/w of Compound A;
about 15% w/w of hydroxypropyl methylcellulose;
about 3% w/w of D-α-tocopheryl polyethylene glycol succinate;
about 17% w/w of microcrystalline cellulose;
about 8.8% w/w of lactose monohydrate;
about 12% w/w of croscarmellose sodium;
about 1% w/w of silicon dioxide; and
about 1.5% w/w of sodium stearyl fumarate.

In embodiments, the solid oral dosage form (e.g., the tablet) comprises:
about 20% w/w of Compound A;
about 28% w/w of hydroxypropyl methylcellulose;
about 2.5% w/w of D-α-tocopheryl polyethylene glycol succinate;
about 27% w/w of microcrystalline cellulose;
about 9% w/w of lactose monohydrate;
about 12% w/w of croscarmellose sodium;
about 1% w/w of silicon dioxide; and
about 1.5% w/w of sodium stearyl fumarate.

In embodiments, the solid oral dosage form (e.g., the tablet) comprises an intra-granular portion and an extra-granular portion, and, optionally, a film coating,
wherein the intra-granular portion comprises:
   about 15% to about 50% w/w of Compound A;
   about 10% to about 40% w/w of hydroxypropyl methylcellulose;
   about 0.5% to about 5% w/w of D-α-tocopheryl polyethylene glycol succinate;
   about 5% to about 10% w/w of microcrystalline cellulose;
   about 5% to about 10% w/w of lactose monohydrate;
   about 1% to about 10% w/w of croscarmellose sodium;
   about 0% to about 5% w/w of silicon dioxide; and
   about 0% to about 2% w/w of sodium stearyl fumarate,
and wherein the extra-granular portion comprises:
   about 5 to about 25% w/w of microcrystalline cellulose;
   about 0% to about 10% w/w of croscarmellose sodium; and
   about 0% to about 2% w/w of sodium stearyl fumarate;
      wherein weight percentages are relative to the total uncoated tablet weight.

In embodiments, the solid oral dosage form (e.g., the tablet) comprises an intra-granular portion and an extra-granular portion, and optionally, a film coating,
wherein the intra-granular portion comprises:
   about 42% w/w of Compound A;
   about 14% w/w hydroxypropyl methylcellulose;
   about 3% w/w d-α-tocopheryl polyethylene glycol succinate;
   about 9% w/w microcrystalline cellulose;
   about 9% w/w lactose monohydrate;
   about 6% w/w croscarmellose sodium;
   about 1% w/w silicon dioxide; and
   about 0.75% w/w sodium stearyl fumarate;
and wherein the extra-granular portion comprises:
   about 8% w/w microcrystalline cellulose;
   about 6% w/w croscarmellose sodium; and
   about 0.75% w/w sodium stearyl fumarate;
      wherein weight percentages are relative to the total uncoated tablet weight.

In embodiments, the solid oral dosage form (e.g., the tablet) comprises an intra-granular portion and an extra-granular portion, and, optionally, a film coating,
wherein the intra-granular portion comprises:
   about 20% w/w of Compound A;
   about 28% w/w of hydroxypropyl methylcellulose;
   about 2.5% w/w of D-α-tocopheryl polyethylene glycol succinate;
   about 8% w/w of microcrystalline cellulose;
   about 9% w/w of lactose monohydrate;
   about 6% w/w of croscarmellose sodium;
   about 1% w/w of silicon dioxide; and
   about 0.75% w/w of sodium stearyl fumarate,
and wherein the extra-granular portion comprises:
   about 18% w/w of microcrystalline cellulose;
   about 6% w/w of croscarmellose sodium; and
   about 0.75% w/w of sodium stearyl fumarate;
      wherein weight percentages are relative to the total uncoated tablet weight.

In embodiments, the solid oral dosage form (e.g., the tablet) comprises about 5 mg, about 10 mg, about 15 mg, about 10 mg, about 25 mg, about 30 mg, about 50 mg, about 75 mg, about 100 mg, about 125 mg, about 150 mg, about 200 mg, about 250 mg, or about 300 mg of Compound A. In embodiments, the solid oral dosage form comprises about 200 mg of Compound A. In embodiments, the solid oral dosage form comprises about 100 mg of Compound A. In embodiments, the solid oral dosage form comprises about 250 mg of Compound A. In embodiments, the solid oral dosage form Comprises about 50 mg of Compound A.

Also disclosed herein are mixtures comprising Compound A, or a pharmaceutically acceptable salt thereof, a polymer, and a surfactant.

Further disclosed herein, are sprayed dried dispersions comprising Compound A, or a pharmaceutically acceptable salt thereof, a polymer, and a surfactant.

Methods of making the spray dried dispersions disclosed herein are also provided. Such methods comprise:
Dissolving Compound A, the polymer, and the surfactant in a solvent to afford a solution comprising Compound A;
Introducing the solution comprising Compound A into a spray dryer;
Spraying the solution comprising Compound A from the spray dryer to form a dispersion of Compound A; and
Optionally, removing the residual solvent from the dispersion of Compound A.

In embodiments of the method, the solvent is a mixture of dichloromethane and methanol. In embodiments the solvent is a mixture of about 90:10 (w/w) to about 70:30 (w/w) dichloromethane:methanol. In embodiments, the solvent is a mixture of about 80:20 (w/w) dichloromethane:methanol. In embodiments, the solvent is a mixture of about 85:15 (w/w) dichloromethane:methanol.

In embodiments of the method, removing residual solvent comprises drying (e.g., agitated conical drying).

### BRIEF DESCRIPTION OF THE DRAWINGS

FIG. 1 shows the results of pharmacokinetic (PK) studies in fasted female dogs orally administered aqueous suspensions of Compound A, as described in Example 2. For formulations B-F, improvement in bioavailability relative to formulation A is indicated.

FIGS. 2A-2D show the results of PK studies in fasted female dogs orally administered tablets of Compound A, as described in Example 3.

FIGS. 3A-3D show the results of PK studies in fed female dogs orally administered tablets of Compound A, as described in Example 3.

### DETAILED DESCRIPTION

The present invention may be understood more readily by reference to the following detailed description of the embodiments of the invention and the Examples included herein. It is to be also understood that the terminology used herein is for the purpose of describing specific embodiments only and is not intended to be limiting.

### (S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione or (3S)-3-[1,3-dihydro-1-oxo-5-[4-[[1-[4-[(1R,2S)-1,2,3,4-tetrahydro-6-hydroxy-2-phenyl-1-naphthalenylphenyl]-4-piperidinyl]methyl]-1-piperazinyl]-2H-isoindol-2-yl]-2,6-piperidinedione (Referred to Herein as “Compound A” or “Cpd A”)

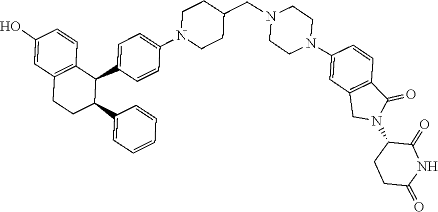 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

is under development as a PROTAC® protein degrader that targets ER for the potential treatment of breast cancer and has been shown to be a useful modulator of targeted protein ubiquitination and degradation via the ubiquitin-proteasome pathway.

Compound A and pharmaceutically acceptable salts thereof are disclosed in International Publication No. WO 2018/102725 and U.S. Pat. Nos. 10,647,698, 10,899,742 and 11,104,666; International Publication No. WO 2021/041348; U.S. Ser. No. 17/472,847; U.S. Ser. No. 17/548,842; and U.S. Ser. No. 17/873,748. The contents of each of the foregoing references are incorporated herein by reference in their entirety.

### DEFINITIONS

Unless otherwise defined herein, scientific, and technical terms used in connection with the present invention have the meanings that are commonly understood by those of ordinary skill in the art.

The invention described herein suitably may be practiced in the absence of any element(s) not specifically disclosed herein.

As used herein, the singular form “a,” “an,” and “the” include plural references unless indicated otherwise. For example, “an” excipient includes one or more excipients.

As used herein, the term “about” when used to modify a numerically defined parameter (e.g., the dose of a compound) means that the parameter may vary by as much as 10% below or above the stated numerical value for that parameter. For example, a dose of about 5 mg means 5 mg 10%, i.e., it may vary from 4.5 mg to 5.5 mg.

As used herein, terms, including, but not limited to, “agent,” “composition,” “compound,” “drug,” and “therapeutic agent” may be used interchangeably to refer to compounds included in the methods and uses of the present disclosure.

The term “amorphous” form refers to solids of disordered arrangements of molecules and that do not possess a distinguishable crystal lattice.

Compound A has the structure:

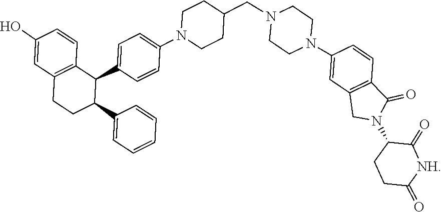 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Compound A is a Biopharmaceutics Classification System Class IV compound (low solubility/low permeability). Compound A may interconvert to its epimer, Compound B:

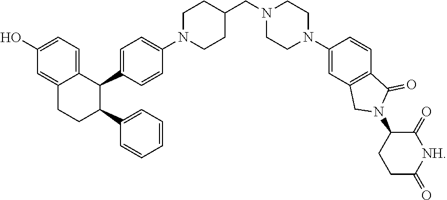 [SMILES: O=C1CC[C@@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

Without wishing to be bound by theory, preclinical data indicates that the exposure of Compound B is limited compared to Compound A (<26%). Evidence indicates that Compound B does not degrade the ER; however, Compound B shows similar antagonism of ER dependent transcription compared to Compound A.

Other embodiments relate to the pharmaceutically acceptable salts of the compounds described herein. Pharmaceutically acceptable salts of the compounds described herein include the acid addition and base addition salts thereof.

Other embodiments also relate to the pharmaceutically acceptable acid addition salts of the compounds described herein. Suitable acid addition salts are formed from acids which form non-toxic salts. Non-limiting examples of suitable acid addition salts, i.e., salts containing pharmacologically acceptable anions, include, but are not limited to, the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of the compounds described herein. Suitable base addition salts are formed from bases that form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine, and zinc salts.

The compounds described herein that are basic in nature can form a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds described herein that include a basic moiety, such as an amino group, may form pharmaceutically acceptable salts with various amino acids, in addition to the acids mentioned above.

The chemical bases that may be used as reagents to prepare pharmaceutically acceptable base salts of those compounds of the compounds described herein that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate, and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

As used herein, the terms, “subject” and “patient,” are used interchangeably, to refer to any animal, including mammals. Mammals according to the disclosure include canine, feline, bovine, caprine, equine, ovine, porcine, rodents, lagomorphs, primates, humans, and the like, and encompass mammals in utero. In embodiments, humans are suitable subjects. Human subjects may be of any gender and at any stage of development.

When formulating a compound into a tablet or other solid oral dosage form, it is desirable to develop a formulation that is storage stable at temperatures and relative humidity levels above those typically encountered. Other desirable properties in a formulation also may be sought, such as fast dissolution so that the tablet quickly dissolves, and the drug is available for absorption. Accordingly, good storage stability and fast dissolution were, inter alia, features that were sought as desirable characteristics for the instant invention.

Active pharmaceutical Ingredients (API) refer to the active ingredient contained in any drug. Any active ingredient is a substance that directly affects the ailment or targets the disease and is intended to use in manufacturing is known as API. These substances are considered crucial as they provide pharmacological activity or direct effects in the diagnosis, cure, treatment, prevention, or affect the body's structure.

“Oral dosage form” as used herein refers to a pharmaceutical drug product that contains a specified amount (dose) of a compound of the disclosure as the API, or a pharmaceutically acceptable salt and/or solvate thereof, and inactive components (excipients), formulated into a particular configuration that is suitable for oral administration, such as an oral tablet, liquid, or capsule. In some embodiments, the oral dosage form comprises a tablet. In some embodiments, the oral dosage form comprises a tablet that can be scored. In some embodiments, the oral dosage form comprises a sublingual tablet.

“Oral administration” as used herein refers to enteral, buccal, sublabial, or sublingual medications in the form of tablets, capsules, syrups, powders, granules, pastilles, solutions, tinctures, elixirs, emulsions, hydrogels, teas, films, disintegrating tablets, mouthwashes, and others.

Drug dissolution represents a critical factor affecting the rate of systemic absorption. A variety of in vitro methods have been developed for assessing the dissolution properties of pharmaceutical formulations, and dissolution testing is sometimes used as a surrogate for the direct evaluation of drug bioavailability. See, e.g., Emmanuel et al., Pharmaceutics (2010), 2:351-63, and references cited therein. Dissolution testing measures the percentage of API that has been released from a drug product (e.g., tablet) and dissolved in a dissolution medium under controlled testing conditions over a defined period of time. To maintain sink conditions, saturation solubility of the drug in the dissolution media should be at least three times the drug concentration. For low solubility compounds, dissolution may sometimes be determined under non-sink conditions. Dissolution is affected by the properties of the API (e.g., particle size, crystal form, bulk density), the composition of the drug product (e.g., drug loading, excipients), the manufacturing process (e.g., compression forces) and the stability under storage conditions (e.g., temperature, humidity).

Methods for assessing the chemical storage stability of solid dosage forms under accelerated aging conditions have been described in the literature. See, e.g., S. T. Colgan, T. J. Watson, R. D. Whipple, R. Nosal, J. V. Beaman, D. De Antonis, “The Application of Science and Risk Based Concepts to Drug Substance Stability Strategies” J. Pharm. Innov. 7:205-2013 (2012); Waterman K C, Carella A J, Gumkowski M J, et al. Improved protocol and data analysis for accelerated shelf-life estimation of solid dosage forms. Pharm Res 2007; 24(4):780-90; and S. T. Colgan, R. J. Timpano, D. Diaz, M. Roberts, R. Weaver, K. Ryan, K. Fields, G. Scrivens, “Opportunities for Lean Stability Strategies” J. Phann. Innov. 9:259-71 (2014).

A “solid oral dosage form” of the present invention is a pharmaceutically acceptable solid oral dosage form that is safe for oral administration to subjects, where all excipients in the dosage form are pharmaceutically acceptable for use in oral formulations, in other words safe for ingestion. In embodiments, the solid oral dosage form is a tablet.

Solid oral dosage forms include, but are not limited to, immediate release tablets and capsules, controlled-release (CR) tablets and capsules, fast-dissolve dosage forms, chewable dosage forms, sachets, etc. Preferably, the dosage form of the present invention is in the form of a tablet, including monolayer or bilayer tablets.

As used herein, the term “unit dose” or “unit dosage” refers to a physically discrete unit that contains a predetermined quantity of active ingredient calculated to produce a desired therapeutic effect. The unit dose or unit dosage may be in the form of a tablet, capsule, sachet, etc. referred to herein as a “unit dosage form.”

The term “fasted” as used herein is defined as follows: a dosing state which is defined following an overnight fast (wherein 0 caloric intake has occurred) of at least 10 hours. Dosing gavage tubes were flushed with 10 mL water. Food was provided 2 hours post dose. Drinking water may be allowed as desired.

The term “fed” as used herein is defined as follows: a dosing state which is defined following an overnight fast (wherein 0 caloric intake has occurred) of at least 10 hours. Food was provided 0.5-1 h before dosing. Gavage tubes were flushed with 10 mL water. Drinking water may be allowed as desired.

The term “dry granulation” means the process of blending bulk active product with at least one excipient. The blend is then compressed, or compacted, to form a compressed material or “compact.” This material may be broken apart to form granules by crushing, grinding, or cutting into dry granulated particles. Optionally, the particles may be further processed. Crushing, grinding, or cutting processes involve an operation that reduces the size of the compressed material such as accomplished by milling or by other operations known to those skilled in the art.

As used herein, the expression “% w/w” means the weight percentage of the component in respect to the total weight of the composition. It is understood that, when a composition is described in which ranges are provided for multiple components, the total amount of all recited components does not exceed 100%. It is within the ability of a skilled artisan to adjust the amounts within the ranges provided to accomplish this.

Unless otherwise specified, the term “solid dispersion,” as used herein, refers to a solid state that comprises at least two constituents, wherein one constituent is homogenously dispersed substantially evenly throughout the other constituent or constituents. It includes solid or glassy solutions, i.e., the dispersion of the constituents is in such a way that the composition is chemically and physically homogenous in nature. In embodiments, the first constituent is an API, and the second constituent is a matrix that comprises a polymer, wherein the API is dispersed significantly uniformly within the matrix (the polymer). The API may be present in an amorphous state or in fine crystalline dispersed form. Also, the API may be available as a mixture of amorphous and crystalline forms. A solid dispersion may comprise more than two constituents. For example, two or more APIs may be dispersed into the matrix, and/or the matrix may comprise two or more polymers. Without limitation, solid dispersions may be physically classified as a eutectic mixture, a solid solution, a glass solution, or suspension, an amorphous precipitate in a glassy or crystalline carrier, a complex, a complexed formation or a combination of the different systems. Solid dispersions may be prepared using various techniques known to those skilled in the art, such as by co-dissolving the API and polymer in a solvent then spray-drying, spray-congealing, evaporation, curing or microwaving, blending and direct compression, mechanical admixture at an elevated, but non-melting temperature, wet granulation, extrusion-spheronization, melt fusion, hot melt extrusion, and the like.

### Mixtures of Compound A

Compound A may be prepared according to the methods disclosed in U.S. Pat. No. 10,647,698, which is incorporated herein in its entirety.

Disclosed herein are mixtures comprising Compound A, a polymer, and a surfactant. In embodiments, the mixture is a dispersion. In embodiments, the mixture is a spray dried dispersion.

In embodiments, the polymer is hydroxypropyl methylcellulose (HPMC, also known as hypromellose), a hydroxypropyl methylcellulose derivative, a polyvinylpyrrolidone copolymer, a methacrylic acid copolymer, polyethylene glycol, or a polyethylene glycol derivative. HPMC may further be described by a letter (e.g., E, K) and number (e.g., 3, 5, 15) indicating the viscosity and substitution chemistry of the polymer.

Hydroxypropyl methylcellulose derivatives include, e.g., organic acid esters of HPMC. A hydroxypropyl methylcellulose derivative may be selected from the group consisting of, e.g., hydroxypropyl methylcellulose acetate succinate (HPMCAS), hydroxypropyl methylcellulose phthalate (HPMCP), hydroxypropyl methylcellulose succinate (HPMCS), hydroxypropyl methylcellulose trimellitate (HPMCT), hydroxypropyl methylcellulose acetate phthalate (HPMCAP), and hydroxypropyl methylcellulose acetate maleate (HPMCAM).

“Polyvinylpyrrolidone copolymer,” “copolymer of vinylpyrrolidone,” or “PVP copolymer” refers to copolymers comprising vinylpyrrolidone and one or more other monomers, such as acrylic monomers, styrene, vinyl acetate, and the like. Polyvinylpyrrolidone vinyl acetate copolymer and copovidone (copolymers of vinylpyrrolidone with other vinyl derivatives) are exemplary polyvinylpyrrolidone copolymers and are commercially available from numerous sources. In embodiments, the polyvinylpyrrolidone copolymer has an average molecular weight of about 1,000 Daltons to about 1,000,000 Daltons; or of about 1,000 Daltons to about 500,000 Daltons; or of about 1,000 Daltons to about 200,000. In embodiments, the polyvinylpyrrolidone copolymer has an average molecular weight of about 1,000 Daltons to about 150,000 Daltons. In embodiments, the polyvinylpyrrolidone copolymer has an average molecular weight of about 10,000 Daltons to about 150,000 Daltons. In embodiments, the polyvinylpyrrolidone copolymer has an average molecular weight of about 50,000 Daltons to about 150,000 Daltons. In embodiments, the PVP copolymer is a polyvinylpyrrolidone vinyl acetate copolymer.

“Polyvinylpyrrolidone vinyl acetate copolymer,” “copolymer of polyvinylpyrrolidone,” “vinyl acetate,” and “PVPVA” refer to a class of copolymers of vinylpyrrolidone and vinyl acetate having varying wt. % ratios of vinylpyrrolidone to vinyl acetate, such as of about 30:70 to about 70:30, including about 30:70, about 35:65, about 50:50, about 60:40, and about 70:30. The wt. % ratios of vinylpyrrolidone to vinyl acetate may result in different properties of the copolymer, including the glass transition temperature. Polyvinylpyrrolidone vinyl acetate copolymer is an exemplary pharmaceutically acceptable polymer and thermoplastic polymer. Polyvinylpyrrolidone vinyl acetate copolymer is commercially available from numerous sources.

The term “methacrylic acid copolymer” refers to the class of polymeric compounds described by the formula:

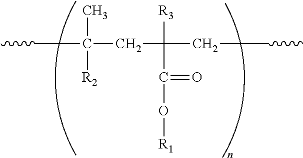

wherein R₁ is alkyl, R₂ is carboxylic acid, and R₃ is H. In embodiments, the methacrylic acid copolymer is a Eudragit® methacrylic acid copolymer.

The term “Eudragit® methacrylic acid copolymer” is used in its conventional sense to refer to copolymers derived from esters of acrylic and methacrylic acid. In embodiments, Eudragit® polymers may be methacrylic acid copolymers (e.g., functional group being carboxylic acid). In embodiments, the Eudragit® polymer is a methacrylic acid Eudragit® polymer, such as Eudragit® L100 or Eudragit® L100-55. Eudragit® polymers are commercially available.

In embodiments, the surfactant is selected from polyethylene glycol, a polyethylene glycol ester, glycerol esters, and mixtures thereof. In embodiments, the surfactant is a vitamin E ester of polyethylene glycol. In embodiments, the surfactant is D-α-tocopheryl polyethylene glycol succinate (i.e., Vitamin E TPGS). In embodiments, the surfactant is D-α-tocopheryl polyethylene glycol 1000 succinate.

In embodiments, the mixture (e.g., the dispersion (e.g., the spray dried dispersion)) comprises Compound A, hydroxypropyl methylcellulose, and D-α-tocopheryl polyethylene glycol succinate.

In embodiments, the mixture (e.g., the dispersion (e.g., the spray dried dispersion)) comprises about 20% (w/w) to about 60% (w/w) Compound A. In embodiments, the mixture (e.g., the dispersion (e.g., the spray dried dispersion)) comprises about 30% (w/w) to about 50% (w/w) Compound A. In embodiments, the mixture (e.g., the dispersion (e.g., the spray dried dispersion)) comprises about 35% (w/w) to about 45% (w/w) Compound A. In embodiments, the mixture (e.g., the dispersion (e.g., the spray dried dispersion)) comprises 20% (w/w) to 60% (w/w) Compound A. In embodiments, the mixture (e.g., the dispersion (e.g., the spray dried dispersion)) comprises 30% (w/w) to 50% (w/w) Compound A. In embodiments, the mixture (e.g., the dispersion (e.g., the spray dried dispersion)) comprises 35% (w/w) to 45% (w/w) Compound A.

In embodiments, the mixture (e.g., the dispersion (e.g., the spray dried dispersion)) comprises about 20% (w/w), about 25% (w/w), about 30% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 50% (w/w), about 55% (w/w), or about 60% (w/w) Compound A.

In embodiments, the mixture (e.g., the dispersion (e.g., the spray dried dispersion)) comprises about 50% (w/w) to about 90% (w/w) Compound A. In embodiments, the mixture (e.g., the dispersion (e.g., the spray dried dispersion)) comprises about 60% (w/w) to about 80% (w/w) Compound A. In embodiments, the mixture (e.g., the dispersion (e.g., the spray dried dispersion)) comprises about 65% (w/w) to about 75% (w/w) Compound A. In embodiments, the mixture (e.g., the dispersion (e.g., the spray dried dispersion)) comprises 50% (w/w) and 90% (w/w) Compound A. In embodiments, the mixture (e.g., the dispersion (e.g., the spray dried dispersion)) comprises 60% (w/w) and 80% (w/w) Compound A. In embodiments, the mixture (e.g., the dispersion (e.g., the spray dried dispersion)) comprises 65% (w/w) and 75% (w/w) Compound A.

In embodiments, the mixture (e.g., the dispersion (e.g., the spray dried dispersion)) comprises about 50% (w/w), about 55% (w/w), about 60% (w/w), about 65% (w/w), about 66% (w/w), about 67% (w/w), about 68% (w/w), about 69% (w/w), about 70% (w/w), about 71% (w/w), about 72% (w/w), about 73% (w/w), about 74% (w/w), about 75% (w/w), about 80% (w/w), about 85% (w/w), or about 90% (w/w) Compound A.

In embodiments, the mixture (e.g., the dispersion (e.g., the spray dried dispersion)) comprises about 35% (w/w) to about 75% (w/w) of a polymer. In embodiments, the mixture (e.g., the dispersion (e.g., the spray dried dispersion)) comprises about 45% (w/w) to about 65% (w/w) of a polymer. In embodiments, the mixture (e.g., the dispersion (e.g., the spray dried dispersion)) comprises about 40% (w/w) to about 50% (w/w) of a polymer. In embodiments, the mixture (e.g., the dispersion (e.g., the spray dried dispersion)) comprises 35% (w/w) to 75% (w/w) of a polymer. In embodiments, the mixture (e.g., the dispersion (e.g., the spray dried dispersion)) comprises 45% (w/w) to 65% (w/w) of a polymer. In embodiments, the mixture (e.g., the dispersion (e.g., the spray dried dispersion)) comprises from 40% (w/w) to 50% (w/w) of a polymer.

In embodiments, the mixture (e.g., the dispersion (e.g., the spray dried dispersion)) comprises about 35% (w/w), about 40% (w/w), about 45% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w), about 60% (w/w), about 65% (w/w), about 70% (w/w), or about 75% (w/w) of a polymer.

In embodiments, the mixture (e.g., the dispersion (e.g., the spray dried dispersion)) comprises about 10% (w/w) to about 40% (w/w) of a polymer. In embodiments, the mixture (e.g., the dispersion (e.g., the spray dried dispersion)) comprises about 15% (w/w) to about 35% (w/w) of a polymer. In embodiments, the mixture (e.g., the dispersion (e.g., the spray dried dispersion)) comprises about 20% (w/w) to about 30% (w/w) of a polymer. In embodiments, the mixture (e.g., the dispersion (e.g., the spray dried dispersion)) comprises 10% (w/w) to 40% (w/w) of a polymer. In embodiments, the mixture (e.g., the dispersion (e.g., the spray dried dispersion)) comprises 15% (w/w) to 35% (w/w) of a polymer. In embodiments, the mixture (e.g., the dispersion (e.g., the spray dried dispersion)) comprises 20% (w/w) to 30% (w/w) of a polymer.

In embodiments, the mixture (e.g., the dispersion (e.g., the spray dried dispersion)) comprises about 10% (w/w), about 15% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 35% (w/w), or about 40% (w/w) of a polymer.

In embodiments, the mixture (e.g., the dispersion (e.g., the spray dried dispersion)) comprises about 35% (w/w) to about 75% (w/w) of hydroxypropyl methylcellulose. In embodiments, the mixture (e.g., the dispersion (e.g., the spray dried dispersion)) comprises about 45% (w/w) to about 65% (w/w) of hydroxypropyl methylcellulose. In embodiments, the mixture (e.g., the dispersion (e.g., the spray dried dispersion)) comprises about 40% (w/w) to about 50% (w/w) of hydroxypropyl methylcellulose. In embodiments, the mixture (e.g., the dispersion (e.g., the spray dried dispersion)) comprises 35% (w/w) to 75% (w/w) of hydroxypropyl methylcellulose. In embodiments, the mixture (e.g., the dispersion (e.g., the spray dried dispersion)) comprises 45% (w/w) to 65% (w/w) of hydroxypropyl methylcellulose. In embodiments, the mixture (e.g., the dispersion (e.g., the spray dried dispersion)) comprises from 40% (w/w) to 50% (w/w) of hydroxypropyl methylcellulose.

In embodiments, the mixture (e.g., the dispersion (e.g., the spray dried dispersion)) comprises about 35% (w/w), about 40% (w/w), about 45% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w), about 60% (w/w), about 65% (w/w), about 70% (w/w), or about 75% (w/w) of hydroxypropyl methylcellulose.

In embodiments, the mixture (e.g., the dispersion (e.g., the spray dried dispersion)) comprises about 10% (w/w) to about 40% (w/w) of hydroxypropyl methylcellulose. In embodiments, the mixture (e.g., the dispersion (e.g., the spray dried dispersion)) comprises about 15% (w/w) to about 35% (w/w) of hydroxypropyl methylcellulose. In embodiments, the mixture (e.g., the dispersion (e.g., the spray dried dispersion)) comprises about 20% (w/w) to about 30% (w/w) of hydroxypropyl methylcellulose. In embodiments, the mixture (e.g., the dispersion (e.g., the spray dried dispersion)) comprises 10% (w/w) to 40% (w/w) of hydroxypropyl methylcellulose. In embodiments, the mixture (e.g., the dispersion (e.g., the spray dried dispersion)) comprises 15% (w/w) to 35% (w/w) of hydroxypropyl methylcellulose. In embodiments, the mixture (e.g., the dispersion (e.g., the spray dried dispersion)) comprises 20% (w/w) to 30% (w/w) of hydroxypropyl methylcellulose.

In embodiments, the mixture (e.g., the dispersion (e.g., a spray dried dispersion)) comprises about 10% (w/w), about 15% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 35% (w/w), or about 40% (w/w) of hydroxypropyl methylcellulose.

In embodiments, the mixture (e.g., the dispersion (e.g., a spray dried dispersion)) comprises about 1% (w/w) to about 10% (w/w) of a surfactant. In embodiments, the mixture comprises about 3% (w/w) to about 8% (w/w) of a surfactant. In embodiments, the mixture comprises about 4% (w/w) to about 6% (w/w) of a surfactant. In embodiments, the mixture comprises from 1% (w/w) to 10% (w/w) of a surfactant. In embodiments, the mixture comprises from 3% (w/w) to 8% (w/w) of a surfactant. In embodiments, the mixture comprises from 4% (w/w) to 6% (w/w) of a surfactant.

In embodiments, the mixture (e.g., the dispersion (e.g., a spray dried dispersion)) comprises about 1% (w/w), about 2% (w/w), about 3% (w/w), about 4% (w/w), about 5% (w/w), about 6% (w/w), about 7% (w/w), about 8% (w/w), about 9% (w/w), or about 10% (w/w) of a surfactant.

In embodiments, the mixture comprises about 1% (w/w) to about 10% (w/w) of D-α-tocopheryl polyethylene glycol succinate. In embodiments, the mixture comprises about 3% (w/w) to about 8% (w/w) of D-α-tocopheryl polyethylene glycol succinate. In embodiments, the mixture comprises about 4% (w/w) to about 6% (w/w) of D-α-tocopheryl polyethylene glycol succinate.

In embodiments, the mixture comprises from 1% (w/w) to 10% (w/w) of D-α-tocopheryl polyethylene glycol succinate. In embodiments, the mixture comprises from 3% (w/w) to 8% (w/w) of D-α-tocopheryl polyethylene glycol succinate. In embodiments, the mixture comprises from 4% (w/w) to 6% (w/w) of D-α-tocopheryl polyethylene glycol succinate.

In embodiments, the mixture (e.g., the dispersion (e.g., the spray dried dispersion)) comprises about 1% (w/w), about 2% (w/w), about 3% (w/w), about 4% (w/w), about 5% (w/w), about 6% (w/w), about 7% (w/w), about 8% (w/w), about 9% (w/w), or about 10% (w/w) of D-α-tocopheryl polyethylene glycol succinate.

In embodiments, the mixture (e.g., the dispersion (e.g., the spray dried dispersion)) comprises:
about 20% (w/w) to about 60% (w/w) of Compound A,
about 35% (w/w) to about 75% (w/w) of a polymer, and
about 1% (w/w) to about 10% (w/w) of a surfactant.

In embodiments, the mixture (e.g., the dispersion (e.g., the spray dried dispersion)) comprises:
about 40% (w/w) of Compound A,
about 55% (w/w) of a polymer, and
about 5% (w/w) of a surfactant.

In some embodiments, the mixture (e.g., the dispersion (e.g., the spray dried dispersion)) comprises:
about 50% to about 90% (w/w) of Compound A,
about 10% to about 40% (w/w) of a polymer, and
about 1% to about 10% (w/w) of a surfactant.

In embodiments, the mixture (e.g., the dispersion (e.g., the spray dried dispersion)) comprises:
about 70% (w/w) Compound A,
about 25% (w/w) of a polymer, and
about 5% (w/w) of a surfactant.

In embodiments, the mixture (e.g., the dispersion (e.g., the spray dried dispersion)) comprises:
about 20% (w/w) to about 60% (w/w) of Compound A,
about 35% (w/w) to about 75% (w/w) of hydroxypropyl methylcellulose, and
about 1% (w/w) to about 10% (w/w) of D-α-tocopheryl polyethylene glycol succinate.

In embodiments, the mixture (e.g., the dispersion (e.g., the spray dried dispersion)) comprises:
about 40% (w/w) of Compound A,
about 55% (w/w) of hydroxypropyl methylcellulose, and
about 5% (w/w) of D-α-tocopheryl polyethylene glycol succinate.

In some embodiments, the mixture (e.g., the dispersion (e.g., the spray dried dispersion)) comprises:
about 50% to about 90% (w/w) of Compound A,
about 10% to about 40% (w/w) of hydroxypropyl methylcellulose, and
about 1% to about 10% (w/w) of D-α-tocopheryl polyethylene glycol succinate.

In embodiments, the mixture (e.g., the dispersion (e.g., the spray dried dispersion)) comprises:
about 70% (w/w) Compound A,
about 25% (w/w) hydroxypropyl methylcellulose, and
about 5% (w/w) D-α-tocopheryl polyethylene glycol succinate.

In embodiments, the mixture (e.g., the dispersion (e.g., the spray dried dispersion)) comprises an amorphous form of Compound A. In embodiments, the mixture (e.g., the spray dried dispersion) comprises a dispersion of amorphous Compound A, hydroxypropyl methylcellulose, and d-α-tocopheryl polyethylene glycol succinate.

### Methods of Preparing Dispersions of Compound A

In embodiments, the mixture of Compound A is in the form of a dispersion. In embodiments, the dispersion is a solid dispersion.

In embodiments, the dispersion is produced by spray drying, lyophilization, hot melt extrusion, milling, solvent evaporation, supercritical fluid processing, or high-shear mixing. Preferably, the dispersion is produced by spray drying.

In embodiments, the dispersion (e.g., the spray dried dispersion) comprises Compound A, a polymer, and a surfactant. In embodiments, the dispersion (e.g., the spray dried dispersion) comprises Compound A, D-α-tocopheryl polyethylene glycol succinate, and hydroxypropyl methylcellulose.

In embodiments, disclosed herein is a method of making the dispersion (e.g., a spray dried dispersion) comprising Compound A disclosed herein. In embodiments, the method comprises:
Dissolving Compound A, a polymer, and a surfactant in a solvent to afford a solution comprising Compound A;
Introducing the solution comprising Compound A into a spray dryer;
Spraying the solution comprising Compound from the spray dryer to form a dispersion (e.g., a spray dried dispersion) of Compound A; and
Optionally, removing the residual solvent from the dispersion (e.g., the spray dried dispersion) of Compound A.

The solvent may be a single organic solvent or a mixture of organic solvents. For example, in embodiments, the solvent is dichloromethane, methanol, or a combination thereof. In embodiments, the solvent is dichloromethane. Alternatively, the solvent is methanol. In embodiments, the solvent is a mixture of dichloromethane and methanol. For example, the ratio of dichloromethane and methanol is about 90:10 (w/w) to about 10:90 (w/w). In embodiments, the ratio of dichloromethane and methanol is about 90:10 (w/w) to about 50:50 (w/w). In embodiments, the ratio of dichloromethane and methanol is about 90:10 (w/w) to about 70:30 (w/w). In embodiments, the ratio of dichloromethane and methanol is about 85:15 (w/w) to about 75:25 (w/w). In embodiments, the ratio of dichloromethane and methanol is 90:10 (w/w) to 70:30 (w/w). In embodiments, the ratio of dichloromethane and methanol is 85:15 (w/w) to 75:25 (w/w). In embodiments, the ratio of dichloromethane and methanol is 70:30 (w/w), about 75:25 (w/w), about 80:20 (w/w), about 85:15 (w/w), or about 90:10 (w/w).

In embodiments, the step of removing residual solvent is accomplished by drying. In embodiments, the drying is accomplished by convection drying, tray drying, filter drying, tumble drying, agitated conical drying, or fluid bed drying. In embodiments, the drying is accomplished by agitated conical drying.

### Dosage Forms of Compound A

In embodiments, the dosage form of Compound A is a solid oral dosage form. In embodiments, the solid oral dosage form is a tablet, a sachet, or a capsule. In embodiments, the solid oral dosage form is a tablet.

Suitable forms for oral administration may include one or more pharmaceutically acceptable excipients, including, for example, carriers, fillers, surfactants, diluents, sweeteners, disintegrants, binders, lubricants, glidants, colorants, flavors, stabilizing agents, coatings, or any mixtures thereof.

Carriers include, but are not limited to, pharmaceutically acceptable excipients and diluents and means a material, composition, or vehicle, such as a liquid or solid filler, diluent, excipient, solvent, or encapsulating material, involved in carrying or transporting a pharmaceutical agent from one organ, or portion of the body, to another organ, or portion of the body of a subject. Examples include, but are not limited to, calcium carbonate, calcium phosphate, various sugars, starches, cellulose derivatives, gelatin, and polymers such as polyethylene glycols.

Fillers include, but are not limited to, mannitol, sucrose, sorbitol, xylitol, microcrystalline cellulose, lactose, silicic acid, silicified microcrystalline cellulose, hydroxypropyl methylcellulose, hydroxypropyl cellulose, starch, pullulan, and fast dissolving carbohydrates such as Pharmaburst™ fast disintegrating tablets, mixtures thereof, and the like. For examples of fast-dissolving carbohydrates see, e.g., U.S. Pat. No. 8,617,588, which is incorporated herein its entirety by reference.

Surfactants include, but are not limited to, non-ionic, anionic, cationic, amphoteric or zwitterionic surfactants. Examples of suitable non-ionic surfactants include ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™; alkyl polyglucosides; stearol ethoxylates; alkyl polyglycosides. Examples of suitable anionic surfactants include alkylether sulfates; alkylether carboxylates; alkyl benzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates. Examples of suitable cationic surfactants include fatty amine salts; fatty diamine salts; quaternary ammonium compounds; phosphonium surfactants; sulfonium surfactants; sulfoxonium surfactants. Examples of suitable zwitterionic surfactants include N-alkyl derivatives of amino acids (such as glycine, betaine, aminopropionic acid); imidazoline surfactants; amine oxides; amidobetaines. Non-limiting examples of a surfactant that may be used in the ospemifene solid dispersions, include, for example. Tween 20, Tween 80, Span 20, Span 80, sodium docusate (e.g., AOT), sodium lauryl sulfate, and poloxamers (e.g., poloxamer 407, Kolliphor® EL, Pluronic F68). Poloxamers are also known by the trade names Synperonics®, Pluronics®, and Kolliphor®/Cremophor®.

Diluents include, but are not limited to, carbohydrates such as monosaccharides like glucose, oligosaccharides like sucrose and lactose (including anhydrous lactose and lactose monohydrate), starch such as maize starch, potato starch, rice starch and wheat starch, pregelatinized starch, calcium hydrogen phosphate, and sugar alcohols like sorbitol, mannitol, erythritol, and xylitol.

Sweeteners include, but are not limited to, sucrose, high fructose corn syrup, fructose, glucose, aspartame, acesulfame K, sucralose, cyclamate, sodium saccharin, neotame, rebaudioside A, and other stevia-based sweeteners.

Disintegrants include, but are not limited to, sodium starch glycolate, sodium carboxymethyl cellulose, calcium carboxymethyl cellulose, croscarmellose sodium, crospovidone, chitosan, agar, alginic acid, calcium alginate, methyl cellulose, microcrystalline cellulose, powdered cellulose, lower alkylsubstituted hydroxypropyl cellulose, hydroxylpropyl starch, low-substituted hydroxypropylcellulose, polacrilin potassium, starch, pregelatinized starch, sodium alginate, magnesium aluminum silicate, polacrilin potassium, povidone, sodium starch glycolate, mixtures thereof, and the like.

Binders include, but are not limited to, hydroxypropylmethylcellulose (HPMC), hydroxypropyl cellulose (HPC), povidone, Copovidone, methylcellulose, powdered acacia, gelatin, gum arabicum, guar gum, carbomer such as carbopol, and polymethacrylates.

Lubricants include, but are not limited to, calcium stearate, glyceryl monostearate, glyceryl behenate, glyceryl palmitostearate, hexagonal boron nitride, hydrogenated vegetable oil, light mineral oil, magnesium stearate, mineral oil, polyethylene glycol, poloxamer, sodium benzoate, sodium lauryl sulfate, sodium stearyl fumarate, stearic acid, talc, zinc stearate, mixtures thereof, and the like.

Glidants include, but are not limited to, silicon dioxide, colloidal silicon dioxide, calcium silicate, magnesium silicate, magnesium trisilicate, talc, starch, mixtures thereof, and the like.

Flavors include, but are not limited to, menthol, peppermint oil, peppermint spirit, vanillin, and almond oil.

In embodiments, the amount of Compound A in the solid oral dosage form (e.g., the tablet) is about 5 mg to about 1000 mg. In embodiments, the amount of Compound A in the solid oral dosage form (e.g., the tablet) is about 5 mg to about 500 mg. In embodiments, the amount of Compound A in the solid oral dosage form (e.g., the tablet) is about 5 mg to about 250 mg. In embodiments, the amount of Compound A in the solid oral dosage form (e.g., the tablet) is about 25 mg to about 250 mg. In embodiments, the amount of Compound A in the solid oral dosage form (e.g., the tablet) is about 25 mg to about 200 mg. In embodiments, the amount of Compound A in the solid oral dosage form (e.g., the tablet) is about 25 mg to about 150 mg. In embodiments, the amount of Compound A in the solid oral dosage form (e.g., the tablet) is about 50 mg to about 150 mg. In embodiments, the amount of Compound A in the solid oral dosage form (e.g., the tablet) is about 75 mg to about 125 mg. In embodiments, the amount of Compound A in the solid oral dosage form (e.g., the tablet) is about 5 mg to about 50 mg. In embodiments, the amount of Compound A in the solid oral dosage form (e.g., the tablet) is about 30 mg to about 40 mg. In embodiments, the amount of Compound A in the solid oral dosage form (e.g., the tablet) is about 65 mg to about 70 mg. In embodiments, the amount of Compound A in the solid oral dosage form (e.g., the tablet) is about 100 mg to about 110 mg. In embodiments, the amount of Compound A in the solid oral dosage form (e.g., the tablet) is about 135 mg to about 145 mg. In embodiments, the amount of Compound A in the solid oral dosage form (e.g., the tablet) is from about 75 mg to about 300 mg. In embodiments, the amount of Compound A in the solid oral dosage form (e.g., the tablet) is from about 100 mg to about 300 mg. In embodiments, the amount of Compound A in the solid oral dosage form (e.g., the tablet) is from about 100 mg to about 250 mg.

In embodiments, the amount of Compound A in the solid oral dosage form (e.g., the tablet) is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 750 mg, or about 1000 mg.

In embodiments, the solid oral dosage form (e.g., the tablet) comprises about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w. 1.8% w/w, about 1.9% w/w, about 2.0% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3.0% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4.0% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5.0% w/w, about 5.5% w/w, about 6.0% w/w, about 6.5% w/w, about 7.0% w/w, about 7.5% w/w, about 8.0% w/w, about 8.5% w/w, about 9.0% w/w, about 9.5% w/w, about 10.0% w/w, about 10.5% w/w, about 11.0% w/w, about 11.5% w/w, about 12.0% w/w, about 12.5% w/w, about 13.0% w/w, about 13.5% w/w, about 14.0% w/w, about 14.5% w/w, about 15.0% w/w, about 15.5% w/w, about 16.0% w/w, about 16.5% w/w, about 17.0% w/w, about 17.5% w/w, about 18.0% w/w, about 18.5% w/w, about 19.0% w/w, about 19.5% w/w, about 2.0% w/w, about 20.5% w/w, about 21.0% w/w, about 21.5% w/w, about 22.0% w/w, about 22.5% w/w, about 23.0% w/w, about 23.5% w/w, about 24.0% w/w, about 24.5% w/w, about 25.0% w/w, about 25.5% w/w, about 26.0% w/w, about 26.5% w/w, about 27.0% w/w, about 27.5% w/w, about 28.0% w/w, about 28.5% w/w, about 29.0% w/w, about 29.5% w/w, about 3.0% w/w, about 30.5% w/w, about 31.0% w/w, about 31.5% w/w, about 32.0% w/w, about 32.5% w/w, about 33.0% w/w, about 33.5% w/w, about 34.0% w/w, about 34.5% w/w, about 35.0% w/w, about 35.5% w/w, about 36.0% w/w, about 36.5% w/w, about 37.0% w/w, about 37.5% w/w, about 38.0% w/w, about 38.5% w/w, about 39.0% w/w, about 39.5% w/w, about 4.0% w/w, about 40.5% w/w, about 41.0% w/w, about 41.5% w/w, about 42.0% w/w, about 42.5% w/w, about 43.0% w/w, about 43.5% w/w, about 44.0% w/w, about 44.5% w/w, about 45.0% w/w, about 45.5% w/w, about 46.0% w/w, about 46.5% w/w, about 47.0% w/w, about 47.5% w/w, about 48.0% w/w, about 48.5% w/w, about 49.0% w/w, about 49.5% w/w, or about 50.0% w/w of Compound A.

In embodiments, the solid oral dosage form (e.g., the tablet) comprises about 1% w/w to about 5% w/w of Compound A, about 2.5% w/w to about 7.5% w/w of Compound A, about 10% w/w to about 15% w/w of Compound A, about 12.5% w/w to about 17.5% w/w of Compound A, about 15% w/w to about 20% w/w of Compound A, about 17.5% w/w to about 22.5% w/w of Compound A, about 20% w/w to about 25% w/w of Compound A, about 22.5% w/w to about 27.5% w/w of Compound A, about 25% w/w to about 30% w/w of Compound A, about 27.5% w/w to about 32.5% w/w of Compound A, about 30% w/w to about 35% w/w of Compound A, about 32.5% w/w to about 37.5% w/w of Compound A, about 35% w/w to about 40% w/w of Compound A, about 37.5% w/w to about 42.5% w/w of Compound A, about 40% w/w to about 45% w/w of Compound A, about 42.5% w/w to about 47.5% w/w of Compound A, about 45% to about 50% w/w of Compound A, about 47.5% w/w to about 52.5% w/w of Compound A, or about 50% w/w to about 55% w/w of Compound A.

In embodiments, the solid oral dosage form (e.g., the tablet) comprises from 1% w/w to 5% w/w of Compound A, from 2.5% w/w to 7.5% w/w of Compound A, from 10% to 15% w/w of Compound A, from 12.5% w/w to 17.5% w/w of Compound A, from 15% w/w to 20% w/w of Compound A, from 17.5% w/w to 22.5% w/w of Compound A, from 20% w/w to 25% w/w of Compound A, from 22.5% w/w to 27.5% w/w of Compound A, from 25% w/w to 30% w/w of Compound A, from 27.5% w/w to 32.5% w/w of Compound A, from 30% w/w to 35% w/w of Compound A, from 32.5% w/w to 37.5% w/w of Compound A, from 35% w/w to 40% w/w of Compound A, from 37.5% w/w to 42.5% w/w of Compound A, from 40% w/w to 45% w/w of Compound A, from 42.5% w/w to 47.5% w/w of Compound A, from 45% w/w to 50% w/w of Compound A, from 47.5% w/w to 52.5% w/w of Compound A, from 50% w/w to 55% w/w of Compound A.

In embodiments, the solid oral dosage form (e.g., the tablet) comprises about 40% w/w to about 70% w/w of a mixture (e.g., a solid dispersion) disclosed herein. In embodiments, the solid oral dosage form (e.g., the tablet) comprises about 40% w/w to about 60% w/w of a mixture (e.g., a solid dispersion) disclosed herein. In embodiments, the solid oral dosage form (e.g., the tablet) comprises about 45% w/w to about 55% w/w of a mixture (e.g., a solid dispersion) disclosed herein. In embodiments, the solid oral dosage form (e.g., the tablet) comprises about 55% w/w to about 65% w/w of a mixture (e.g., a solid dispersion) disclosed herein.

In embodiments, the solid oral dosage form (e.g., the tablet) comprises 40% w/w to 70% w/w of a mixture (e.g., a solid dispersion) disclosed herein. In embodiments, the solid oral dosage form (e.g., the tablet) comprises 40% w/w to 60% w/w of a mixture (e.g., a solid dispersion) disclosed herein. In embodiments, the solid oral dosage form (e.g., the tablet) comprises 45% w/w to 55% w/w of a mixture (e.g., a solid dispersion) disclosed herein. In embodiments, the solid oral dosage form (e.g., the tablet) comprises 55% w/w to 65% w/w of a mixture (e.g., a solid dispersion) disclosed herein.

In some embodiments, the solid oral dosage form (e.g., the tablet) comprises:
about 40% to about 60% w/w of a mixture (e.g., a solid dispersion) disclosed herein;
about 10% to about 40% w/w of microcrystalline cellulose;
about 5% to about 15% w/w of lactose monohydrate;
about 5% to about 15% w/w of croscarmellose sodium;
about 0% to about 5% w/w of silicon dioxide; and
about 0% to about 2% w/w of sodium stearyl fumarate.

In embodiments, the solid oral dosage form (e.g., the tablet) comprises:
40% to 60% w/w of a mixture (e.g., a solid dispersion) disclosed herein;
10% to 40% w/w of microcrystalline cellulose;
5% to 15% w/w of lactose monohydrate;
5% to 15% w/w of croscarmellose sodium;
0% to 5% w/w of silicon dioxide; and
0% to 2% w/w of sodium stearyl fumarate.

In embodiments, the solid oral dosage form (e.g., the tablet) comprises:
about 40% to about 70% w/w of a mixture (e.g., a solid dispersion) disclosed herein
about 10% to about 40% w/w of microcrystalline cellulose;
about 5% to about 15% w/w of lactose monohydrate;
about 5% to about 15% w/w of croscarmellose sodium;
about 0% to about 5% w/w of silicon dioxide; and
about 0% to about 2% w/w of sodium stearyl fumarate.

In embodiments, the solid oral dosage form (e.g., the tablet) comprises:
40% to 70% w/w of a mixture (e.g., a solid dispersion) disclosed herein;
10% to 40% w/w of microcrystalline cellulose;
5% to 15% w/w of lactose monohydrate;
5% to 15% w/w of croscarmellose sodium;
0% to 5% w/w of silicon dioxide; and
0% to 2% w/w of sodium stearyl fumarate.

In embodiments, the solid oral dosage form (e.g., the tablet) comprises:
about 45% to about 55% w/w of a mixture (e.g., a solid dispersion) disclosed herein (e.g., a mixture (e.g., a solid dispersion) comprising Compound A/HPMC/TPGS in a ratio of about 40:55:5 (w/w));
about 10% to about 40% w/w of microcrystalline cellulose;
about 5% to about 15% w/w of lactose monohydrate;
about 5% to about 15% w/w of croscarmellose sodium;
about 0% to about 5% w/w of silicon dioxide; and
about 0% to about 2% w/w of sodium stearyl fumarate.

In embodiments, the solid oral dosage form (e.g., the tablet) comprises:
45% to 55% w/w of a mixture (e.g., a solid dispersion) disclosed herein (e.g., a mixture (e.g., a solid dispersion) comprising Compound A/HPMC/TPGS in a ratio of about 40:55:5 (w/w));
10% to 40% w/w of microcrystalline cellulose;
5% to 15% w/w of lactose monohydrate;
5% to 15% w/w of croscarmellose sodium;
0% to 5% w/w of silicon dioxide; and
0% to 2% w/w of sodium stearyl fumarate.

In some embodiments, the solid oral dosage form (e.g., the tablet) comprises:
about 55% to about 65% w/w of a mixture (e.g., a solid dispersion) disclosed herein (e.g., a mixture (e.g., a solid dispersion) comprising Compound A/HPMC/TPGS in a ratio of about 70:25:5 (w/w));
about 10% to about 40% w/w of microcrystalline cellulose;
about 5% to about 15% w/w of lactose monohydrate;
about 5% to about 15% w/w of croscarmellose sodium;
about 0% to about 5% w/w of silicon dioxide; and
about 0% to about 2% w/w of sodium stearyl fumarate.

In embodiments, the solid oral dosage form (e.g., the tablet) comprises:
55% to 65% w/w of a mixture (e.g., the solid dispersion) disclosed herein (e.g., a mixture or dispersion comprising about 70% Compound A by weight, preferably a mixture (e.g., a solid dispersion) comprising Compound A/HPMC/TPGS in a ratio of about 70:25:5 (w/w));
10% to 40% w/w of microcrystalline cellulose;
5% to 15% w/w of lactose monohydrate;
5% to 15% w/w of croscarmellose sodium;
0% to 5% w/w of silicon dioxide; and
0% to 2% w/w of sodium stearyl fumarate.

In embodiments, the solid oral dosage form (e.g., the tablet) comprises:
about 50% w/w of a mixture (e.g., the solid dispersion) disclosed herein (e.g., a mixture (e.g., a solid dispersion) comprising about 70% Compound A by weight, preferably a mixture (e.g., a solid dispersion) comprising Compound A/HPMC/TPGS in a ratio of about 40:55:5 (w/w));
about 27% w/w of microcrystalline cellulose;
about 9% w/w of lactose monohydrate;
about 12% w/w of croscarmellose sodium;
about 1% w/w of silicon dioxide; and
about 1.5% w/w of sodium stearyl fumarate.

In embodiments, the solid oral dosage form (e.g., the tablet) comprises:
about 60% w/w of a mixture (e.g., the solid dispersion) disclosed herein (e.g., a mixture (e.g., a solid dispersion) comprising about 70% Compound A by weight, preferably a mixture (e.g., a solid dispersion) comprising Compound A/HPMC/TPGS in a ratio of about 70:25:5 (w/w));
about 17% w/w of microcrystalline cellulose;
about 8.8% w/w of lactose monohydrate;
about 12% w/w of croscarmellose sodium;
about 1% w/w of silicon dioxide; and
about 1.5% w/w sodium stearyl fumarate.

In embodiments, the solid oral dosage form (e.g., the tablet) comprises an intra-granular portion and an extra-granular portion, and, optionally, a film coating, wherein the intra-granular portion comprises:
about 42% w/w of Compound A;
about 14% w/w hydroxypropyl methylcellulose;
about 3% w/w D-α-tocopheryl polyethylene glycol succinate;
about 9% w/w microcrystalline cellulose;
about 9% w/w lactose monohydrate;
about 6% w/w croscarmellose sodium;
about 1% w/w silicon dioxide; and
about 0.75% w/w sodium stearyl fumarate,
and wherein the extra-granular portion comprises:
about 8% w/w microcrystalline cellulose;
about 6% w/w croscarmellose sodium; and
about 0.75% w/w sodium stearyl fumarate.

In embodiments, the solid oral dosage form (e.g., the tablet) comprises an intra-granular portion and an extra-granular portion, and, optionally, a film coating, wherein the intra-granular portion comprises:
about 40% to about 70% w/w of a mixture (e.g., a solid dispersion) as disclosed herein.
about 5% to about 10% w/w of microcrystalline cellulose;
about 5% to about 10% w/w of lactose monohydrate;
about 1% to about 10% w/w of croscarmellose sodium;
about 0% to about 5% w/w of silicon dioxide; and
about 0% to about 2% w/w of sodium stearyl fumarate,
and wherein the extra-granular portion comprises:
about 5% to about 25% w/w of microcrystalline cellulose;
about 0% to about 10% w/w of croscarmellose sodium; and
about 0% to about 2% w/w of sodium stearyl fumarate.

In embodiments, the solid oral dosage form (e.g., the tablet) comprises an intra-granular portion and an extra-granular portion, and, optionally, a film coating, wherein the intra-granular portion comprises:
40% to 70% w/w of a mixture (e.g., a solid dispersion) as disclosed herein.
5% to 10% w/w of microcrystalline cellulose;
5% to 10% w/w of lactose monohydrate;
1% to 10% w/w of croscarmellose sodium;
0% to 5% w/w of silicon dioxide; and
0% to 2% w/w of sodium stearyl fumarate,
and wherein the extra-granular portion comprises:
5% to 25% w/w of microcrystalline cellulose;
0% to 10% w/w of croscarmellose sodium; and
0% to 2% w/w of sodium stearyl fumarate.

In embodiments, the solid oral dosage form (e.g., the tablet) comprises an intra-granular portion and an extra-granular portion, and, optionally, a film coating, wherein the intra-granular portion comprises:
about 40% to about 60% w/w of a mixture (e.g., solid dispersion) as disclosed herein;
about 5% to about 10% w/w of microcrystalline cellulose;
about 5% to about 10% w/w of lactose monohydrate;
about 1% to about 10% w/w of croscarmellose sodium;
about 0% to about 5% w/w of silicon dioxide; and
about 0% to about 2% w/w of sodium stearyl fumarate,
and wherein the extra-granular portion comprises
about 5% to about 25% w/w of microcrystalline cellulose;
about 0% to about 10% w/w of croscarmellose sodium; and
about 0% to about 2% w/w of sodium stearyl fumarate.

In embodiments, the solid oral dosage form (e.g., the tablet) comprises an intra-granular portion and an extra-granular portion, and, optionally, a film coating, wherein the intra-granular portion comprises:
40% to 60% w/w of a mixture and/or solid dispersion as disclosed herein.
5% to 10% w/w microcrystalline cellulose;
5% to 10% w/w lactose monohydrate;
1% to 10% w/w croscarmellose sodium;
0% to 5% w/w silicon dioxide; and
0% to 2% w/w sodium stearyl fumarate,
and wherein the extra-granular portion comprises
5% to 25% w/w microcrystalline cellulose;
0% to 10% w/w croscarmellose sodium; and
0% to 2% w/w sodium stearyl fumarate.

In some embodiments, the solid oral dosage form (e.g., the tablet) comprises an intra-granular portion and an extra-granular portion, and, optionally, a film coating, wherein the intra-granular portion comprises:
about 50% w/w of a mixture (e.g., a solid dispersion) as disclosed herein (e.g., a mixture (e.g., a solid dispersion) comprising about 70% Compound A by weight, preferably a mixture (e.g., a solid dispersion) comprising Compound A/HPMC/TPGS in a ratio of about 40:55:5 (w/w));
about 8% w/w microcrystalline cellulose;
about 9% w/w lactose monohydrate;
about 6% w/w croscarmellose sodium;
about 1% w/w silicon dioxide; and
about 0.75% w/w sodium stearyl fumarate,
and wherein the extra-granular portion comprises
about 18% w/w microcrystalline cellulose;
about 6% w/w croscarmellose sodium; and
about 0.75% w/w sodium stearyl fumarate.

In embodiments, the solid oral dosage form (e.g., the tablet) comprises an intra-granular portion and an extra-granular portion, and, optionally, a film coating, wherein the intra-granular portion comprises:
about 60% w/w of a mixture (e.g., a solid dispersion) as disclosed herein (e.g., a mixture (e.g., a solid dispersion) comprising about 70% Compound A by weight, preferably a mixture (e.g., a solid dispersion) comprising Compound A/HPMC/TPGS in a ratio of about 70:25:5 (w/w));
about 9% w/w microcrystalline cellulose;
about 9% w/w lactose monohydrate;
about 6% w/w croscarmellose sodium;
about 1% w/w silicon dioxide; and
about 0.75% w/w sodium stearyl fumarate,
and wherein the extra-granular portion comprises:
about 8% w/w microcrystalline cellulose;
about 6% w/w croscarmellose sodium; and
about 0.75% w/w sodium stearyl fumarate.

In embodiments, the solid oral dosage form (e.g., the tablet) comprises:
about 15% w/w to about 50% w/w of Compound A;
about 10% w/w to about 40% w/w of hydroxypropyl methylcellulose;
about 0.5% w/w to about 5% w/w of D-α-tocopheryl polyethylene glycol succinate;
about 10% w/w to about 40% w/w of microcrystalline cellulose;
about 5% w/w to about 15% w/w of lactose monohydrate;
about 5% w/w to about 15% w/w of croscarmellose sodium;
about 0% w/w to about 5% w/w of silicon dioxide; and
about 0% w/w to about 2% w/w of sodium stearyl fumarate.

In embodiments, the solid oral dosage form (e.g., the tablet) comprises:
15% to 50% w/w of Compound A;
10% to 40% w/w of hydroxypropyl methylcellulose;
0.5% to 5% w/w of D-α-tocopheryl polyethylene glycol succinate;
10% to 40% w/w of microcrystalline cellulose;
5% to 15% w/w of lactose monohydrate;
5% to 15% w/w of croscarmellose sodium;
0% to 5% w/w of silicon dioxide; and
0% to 2% w/w of sodium stearyl fumarate.

In embodiments, the solid oral dosage form (e.g., the tablet) comprises:
about 20% w/w of Compound A;
about 28% w/w of hydroxypropyl methylcellulose;
about 2.5% w/w of D-α-tocopheryl polyethylene glycol succinate;
about 27% w/w of microcrystalline cellulose;
about 9% w/w of lactose monohydrate;
about 12% w/w of croscarmellose sodium;
about 1% w/w of silicon dioxide; and
about 1.5% w/w of sodium stearyl fumarate.

In embodiments, the solid oral dosage form (e.g., the tablet) comprises:
about 42% w/w of Compound A;
about 15% w/w of hydroxypropyl methylcellulose;
about 3% w/w of D-α-tocopheryl polyethylene glycol succinate;
about 17% w/w of microcrystalline cellulose;
about 8.8% w/w of lactose monohydrate;
about 12% w/w of croscarmellose sodium;
about 1% w/w of silicon dioxide; and
about 1.5% w/w of sodium stearyl fumarate.

In embodiments, the solid oral dosage form (e.g., the tablet) comprises an intra-granular portion and an extra-granular portion, and, optionally, a film coating,
wherein the intra-granular portion comprises:
   about 15% to about 50% w/w of Compound A;
   about 10% to about 40% w/w of hydroxypropyl methylcellulose;
   about 0.5% to about 5% w/w of D-α-tocopheryl polyethylene glycol succinate;
   about 5% to about 10% w/w of microcrystalline cellulose;
   about 5% to about 10% w/w of lactose monohydrate;
   about 1% to about 10% w/w of croscarmellose sodium;
   about 0% to about 5% w/w of silicon dioxide; and
   about 0% to about 2% w/w of sodium stearyl fumarate,
and wherein the extra-granular portion comprises:
   about 5% to about 25% w/w of microcrystalline cellulose;
   about 0% to about 10% w/w of croscarmellose sodium; and
   about 0% to about 2% w/w of sodium stearyl fumarate;
      wherein weight percentages are relative to the total uncoated tablet weight.

In embodiments, the solid oral dosage form (e.g., the tablet) comprises an intra-granular portion and an extra-granular portion, and, optionally, a film coating,
wherein the intra-granular portion comprises:
   about 20% w/w of Compound A;
   about 28% w/w of hydroxypropyl methylcellulose;
   about 2.5% w/w of D-α-tocopheryl polyethylene glycol succinate;
   about 8% w/w of microcrystalline cellulose;
   about 9% w/w of lactose monohydrate;
   about 6% w/w of croscarmellose sodium;
   about 1% w/w of silicon dioxide; and
   about 0.75% w/w of sodium stearyl fumarate,
and wherein the extra-granular portion comprises:
   about 18% w/w of microcrystalline cellulose;
   about 6% w/w of croscarmellose sodium; and
   about 0.75% w/w of sodium stearyl fumarate;
      wherein weight percentages are relative to the total uncoated tablet weight.

In embodiments, the solid oral dosage form (e.g., the tablet) comprises an intra-granular portion and an extra-granular portion, and optionally, a film coating,
wherein the intra-granular portion comprises:
   about 42% w/w of Compound A;
   about 14% w/w hydroxypropyl methylcellulose;
   about 3% w/w D-α-tocopheryl polyethylene glycol succinate;
   about 9% w/w microcrystalline cellulose;
   about 9% w/w lactose monohydrate;
   about 6% w/w croscarmellose sodium;
   about 1% w/w silicon dioxide; and
   about 0.75% w/w sodium stearyl fumarate;
and wherein the extra-granular portion comprises:
   about 8% w/w microcrystalline cellulose;
   about 6% w/w croscarmellose sodium; and
   about 0.75% w/w sodium stearyl fumarate;
      wherein weight percentages are relative to the total uncoated tablet weight.

### Methods of Preparing Dosage Forms of Compound A

Solid formulations for oral administration may be formulated to be immediate and/or modified release. Modified release formulations include delayed-, sustained-, pulsed-, controlled-, targeted-, and programmed-release. For a general description of modified release formulations, see U.S. Pat. No. 6,106,864.

Pharmaceuticals in the form of solid shaped tablets are typically manufactured by compressing the materials that make up the final product into the desired tablet form. Such materials may include active pharmaceutical ingredients as well as pharmaceutically non-active excipients that impart necessary or useful properties to the product during and after the manufacturing process. Tablet hardness, or tensile strength may be used as a measure of the cohesiveness of the ingredients of a tablet. If a tablet does not possess sufficient cohesive properties, the tablet may fall apart on handling. The final formulation may comprise one or more layers and may be coated or uncoated.

As is known in the art, granulation is a process used to improve the handling and manufacturing properties of a formulation, for example by increasing particle size to improve flow. Granulation does not substantially change the physical form of the drug such as its crystalline or amorphous character. Various processes are used by those of skill in the art for preparing tablet dosage forms. Examples of such processes include dry-granulation, wet-granulation, fluid-bed granulation, and direct compression. The type of method used may depend upon factors such as physical characteristics of the active pharmaceutical ingredients in the formulation, the types of excipients used and the desired physical characteristics of the final product. Each of these processes include steps involving mixing of the ingredients of the dosage form.

Some amount of mixing of the ingredients of a dosage form is usually necessary to have a homogeneous and consistent final product. However, in the preparation of pharmaceutical tablets by wet and dry granulation it has been found that the extent and intensity of the mixing of the ingredients prior to compression is related to a loss of compressibility and cohesiveness of the formulation, resulting in reduced tablet hardness.

A similar result may be observed when roller compaction is used, for example, in dry granulation methods. Roller compaction may be employed as a method to form the granules that are subsequently compressed into tablets. Roller compaction may reduce the subsequent compressibility and cohesiveness of the dosage form.

Dry granulation is a process in which granulates are formed by a compaction step that is followed by sizing the compacts into particles that may be processed easily. It is often used to improve flow properties and/or densify the formulation which may facilitate further manufacturing processes such as tableting, encapsulation and powder filling. The compacts are made directly from powder blends that usually contain an active ingredient and other excipients including a lubricant.

The use of dry granulation techniques may be preferred to wet granulation methods because of shorter processing times and cost advantages. However, dry granulation is generally limited to those situations in which the drug or active ingredient has physical characteristics suitable for forming pharmaceutically acceptable granulations and dosage forms such as tablets.

The addition of at least one excipient to the formulation is generally required and will contribute to increasing the tablet size of the final product. As tablet size must be within certain parameters to function as a suitable dosage form, there is a limit beyond which increasing tablet size to accommodate increasing amounts of excipients to enhance compatibility is not practical. As a result, manufacturers are often limited to using the dry granulation method for formulations containing a low dose of the active ingredient per compressed tablet such that the formulation may accommodate sufficient levels of excipient to make dry granulation practical.

In the development of pharmaceutical dosage forms, it is important to balance several different objectives. It is important to prepare a pharmaceutical dosage form as economically as possible. It would be desirable to have a simple production method comprising a few processing steps. The dosage form should also optimally make available the active compound contained therein to the patient. Further, the dosage form should be easy to swallow. Smaller dosage forms may be better accepted by patients and may improve patient compliance.

The final pharmaceutical composition is processed into a unit dosage form (e.g., tablet or capsule) and then packaged for distribution. The processing step will vary depending upon the particular unit dosage form. For example, a tablet is generally compressed under pressure into a desired shape and a capsule employs a simple fill operation. Those skilled in the art are aware of the procedures used for manufacturing the various unit dosage forms.

Tablets are typically formed by pressure being applied to the material to be tableted on a tablet press. A formulation must have good flow properties for precise volumetric feeding of the material to the die cavity and suitable compressibility, compatibility, and ejection properties to form a tablet.

There are several tablet presses, each varying in productivity but similar in basic function and operation. All compress a tablet formulation within a die cavity by pressure exerted between two steel punches, a lower punch, and an upper punch. Tablet presses are typically designed to have a hopper for holding and feeding the formulation, a feeding mechanism for feeding the formulation to the die cavity, provision for placement of punches and dies, and in rotary tablet presses a cam track for guiding the movement of the punches. Two types of tablet presses are the single station or single-punch press and the multistation rotary press. Some tablet presses provide longer dwell times than others, allowing increased bonding to occur. Other presses may provide precompression.

Wet granulation methods may also be employed for preparing the granules of the pharmaceutical composition. Wet granulation methods are described in Remington: The Science and Practice of Pharmacy, Mack Publishing Company, Easton, Pa., 19th Edition 1995. These and other methods are generally known by those skilled in the art. If wet granulation is employed, a volatilizable agent may be incorporated in the mixture before, during or after mixing of the ingredients, but prior to formation of granules. For example, a solid volatilizable agent may be blended with the powders of the mixture prior to, during or after the addition of binding agent solutions. Other solid dosage forms may be prepared using techniques including rotary bed granulation or spray-dried dispersion (SDD).

In embodiments, using standard coating procedures, such as those described in Remingtons's Pharmaceutical Sciences, 20th Edition (2000), a film coating is provided around the solid oral dosage form (e.g., the tablet) of Compound A. In embodiments, some or all the particles of the solid oral dosage form (e.g., the tablet) of Compound A are coated. In embodiments where a film coating is provided around the solid oral dosage form (e.g., the tablet) of Compound A, all weight percentages are relative to the total uncoated tablet weight.

Alternatively, some or all the particles of the solid oral dosage form (e.g., the tablet) of Compound A are microencapsulated. In embodiment, the particles of the solid oral dosage form (e.g., the tablet) of Compound A are not microencapsulated and are uncoated.

### Methods of Treatment

In embodiments, provided herein are methods for treating cancer in a subject comprising administering to the subject a solid oral dosage form comprising an effective amount of Compound A as described herein.

Solid oral dosage forms comprising Compound A may be administered alone or in combination with other drugs, in particular CDK4/6 inhibitors (e.g., dalpiciclib, trilaciclib, lerociclib, AT7519M, dinaciclib, ribociclib, abemaciclib, or palbociclib, or a pharmaceutically acceptable salt thereof).

The term “combination,” as used herein, unless otherwise indicated, refers to the use of Compound A with one or more therapeutic agents, wherein Compound A and the one or more therapeutic agents are administered intermittently, concurrently, or sequentially, according to the same or different route of administration and according to the same or different dosage schedules.

The term “locally advanced,” as used herein, as it relates to cancer, may or may not be treated with curative intent. For example, locally advanced breast cancer (LABC) is defined by the U.S. National Comprehensive Cancer Network as a subset of breast cancer characterized by the most advanced breast tumors in the absence of distant metastasis, wherein the tumors are more than 5 cm in size with regional lymphadenopathy; tumors of any size with direct extension to the chest wall or skin, or both (including ulcer or satellite nodules), regardless of regional lymphadenopathy; presence of regional lymphadenopathy (clinically fixed or matted axillary lymph nodes, or any of infraclavicular, supraclavicular, or internal mammary lymphadenopathy) regardless of tumor stage. (Garg et al. Curr Oncol. 2015 October; 22(5): e409-e410; National Comprehensive Cancer Network NCCN Clinical Practice Guidelines in Oncology: Breast Cancer. Fort Washington, PA: NCCN; 2015. Ver. 2.2015.)

The term “metastatic” as used herein, as it relates to cancer, cannot be treated with curative intent. For example, metastatic breast cancer refers to breast cancer that has spread beyond the breast and nearby lymph nodes to other parts of the body, e.g., bones, liver, lungs, brain. (www.cancer.org/cancer/breast-cancer.)

Those skilled in the art will be able to recognize and diagnose locally advanced and metastatic cancer in a patient or subject.

For convenience, certain well-known abbreviations may be used herein, including: castration resistant prostate cancer (CRPC), estrogen receptor positive (ER+), human epidermal growth factor receptor 2 negative (HER2−), hormone receptor (HR), human epidermal growth factor receptor 2 positive (HER2+), non-small cell lung cancer (NSCLC), and progesterone receptor (PR).

In embodiments, the cancer is selected from lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, head and neck cancer, and combinations of two or more of the foregoing cancers.

Also disclosed herein are methods of treating cancer in a subject. In embodiments, the methods comprise treating cancer in a subject comprising administering to the subject a solid oral dosage form comprising an amount of Compound A, as described herein, that is effective in treating the cancer.

In embodiments, the cancer is breast, lung, colon, brain, head and neck, prostate, stomach, pancreatic, ovarian, melanoma, endocrine, uterine, testicular, or bladder.

In embodiments, the cancer is breast, lung, prostate, pancreatic, or ovarian.

In embodiments, the cancer is breast, lung, or prostate.

In embodiments, the cancer is breast cancer.

In embodiments, the breast cancer is metastatic breast cancer.

In embodiments, the breast cancer is locally advanced breast cancer.

In embodiments, the breast cancer is HR+ breast cancer.

In embodiments, the HR+ breast cancer is PR+ and/or ER+ breast cancer.

In some embodiments, the breast cancer is PR+ breast cancer.

In embodiments, the breast cancer is ER+ breast cancer.

In embodiments, the breast cancer is ER+ HER2− breast cancer.

In embodiments, the breast cancer is ER+ HER2+ breast cancer.

In embodiments, the breast cancer is locally advanced or metastatic ER+ breast cancer.

In embodiments, the breast cancer is locally advanced or metastatic ER+ HER2− breast cancer.

In embodiments, the breast cancer is locally advanced or metastatic ER+ HER2+ breast cancer.

In embodiments, the breast cancer is metastatic, ER+, HER2− breast cancer.

In embodiments, the breast cancer is metastatic, ER+, HER2− breast cancer that is also locally advanced.

In embodiments, the lung cancer is non-small cell lung cancer.

In embodiments, the lung cancer is locally advanced or metastatic non-small cell lung cancer.

In embodiments, the prostate cancer is CRPC.

In embodiments, the prostate cancer is locally advanced or metastatic CRPC.

Also disclosed herein are methods of treating solid tumors in a subject. In embodiments, disclosed herein are methods of treating solid tumors in a subject comprising administering to the subject a solid oral dosage form, comprising an amount of Compound A, as described herein, that is effective in treating the solid tumor.

In embodiments, the solid tumor is breast, lung, colon, brain, head and neck, prostate, stomach, pancreatic, ovarian, melanoma, endocrine, uterine, testicular, or bladder.

In embodiments, the solid tumor is breast, lung, prostate, pancreatic, or ovarian.

In embodiments, the solid tumor is breast, lung, or prostate.

In embodiments, the solid tumor is breast cancer. For example, in certain embodiments the breast cancer is HR+ breast cancer. In other embodiments, the HR+ breast cancer is PR+ and/or ER+ breast cancer ER+ breast cancer.

In embodiments, the solid tumor is breast cancer. For example, in certain embodiments, the breast cancer is ER+ HER2− breast cancer.

In embodiments, the solid tumor is breast cancer. For example, in certain embodiments, the breast cancer is ER+ HER2+ breast cancer.

In embodiments, the solid tumor is breast cancer. For example, in certain embodiments, the breast cancer is locally advanced or metastatic ER+ HER2− breast cancer.

In embodiments, the solid tumor is breast cancer. For example, in certain embodiments, the breast cancer is locally advanced or metastatic ER+ HER2+ breast cancer.

In embodiments, the solid tumor is lung cancer. For example, in certain embodiments, the lung cancer is non-small cell lung cancer.

In embodiments, the solid tumor is lung cancer. For example, in certain embodiments, the lung cancer is locally advanced or metastatic non-small cell lung cancer.

In embodiments, the solid tumor is prostate cancer. For example, in certain embodiments, the prostate cancer is CRPC.

In embodiments, the solid tumor is prostate cancer. For example, in certain embodiments, the prostate cancer is locally advanced or metastatic castration resistant prostate cancer.

Also disclosed herein are methods of treating hematologic tumors in a subject. In certain embodiments, the method comprises treating hematologic tumors in a subject comprising administering to the subject a solid oral dosage form comprising an amount of Compound A, as described herein, that is effective in treating the hematologic tumor.

In embodiments, the hematologic tumor is leukemia, lymphoma, or multiple myeloma.

In embodiments, the hematologic tumor is leukemia or lymphoma.

Also disclosed herein are methods of treating cancer in a subject with locally advanced or metastatic ER+ HER2− breast cancer, CRPC, or NSCLC whose disease progressed on or is intolerant to standard therapy.

Also disclosed herein are methods of treating cancer in a subject with locally advanced or metastatic ER+ HER2− breast cancer, CRPC, or NSCLC whose disease progressed on or is intolerant to standard therapy.

Also disclosed herein are methods of treating cancer in a subject with locally advanced or metastatic 2L+ ER+ HER2 breast cancer who have received prior hormonal/endocrine therapy and chemotherapy in the locally advanced/metastatic setting.

Also disclosed herein are methods of treating cancer in a subject with locally advanced or metastatic 2L+ ER+ HER2 breast cancer who have received prior treatment with a CDK4/6 inhibitor.

### EXAMPLES

In order that this invention may be better understood, the following examples are set forth. These examples are for purposes of illustration only and are not to be construed as limiting the scope of the invention in any manner.

### Example 1. Preparation of 50 mg, 100 mg, 200 mg, and 250 mg Tablets of Compound A

50 mg, 100 mg, 200 mg, and 250 mg tablets of Compound A were produced using two different formulations resulting in 20% and 42% Compound A drug load. Compound A was prepared in the form of a spray dried solid dispersion (SDD), composed of Compound A/HPMC E3/Vitamin E TPGS in 70:25:5% w/w or 40:55:5% w/w ratio.

Compositions of the 50 mg, 100 mg, 200 mg, and 250 mg tablets of Compound A as an SDD, composed of Compound A/HPMC E3/Vitamin E TPGS, and the composition of the 100 mg tablets of Compound A as an SDD, composed of 40:55:5% w/w ratio, are shown below in Table 2. Materials used in preparation of the spray dried dispersions (SDDs) are shown below in Table 4.

One of ordinary skill in pharmaceutical manufacturing would understand that the values provided in Tables 1-4 are theoretical formulations for a single unit (the QQ formula (Qua Que)). When a pharmaceutical product is scaled up to multiple units, the quantity for each component is multiplied by the number of units required. Due to differences in balance accuracy and tolerances, in calculating back from the weights used to manufacture a batch, there is a potential for slight differences in the QQ values. Additionally, during manufacturing, certain limits are allowed for the weights provided; however, as a percentage of the weight measured, these values will be the same as the QQ formula. This is recognized within the industry and with the regulators.

TABLE 1
Tablet Compositions for 50 mg Tablet

| | 50 mg Tablet |
|---|---|
| Tablet Weight (mg - uncoated) | 120.0 mg |

| | |
|---|---|
| | Percent by weight |
| Component | (% w/w) |

Intra-granular Blend

| SDD (Compound A:HPMC:TPGS 70/25/5) | 59.52% | w/w |
|---|---|---|
| SDD (Compound A:HPMC:TPGS 40/55/5) | — | |
| Avicel Ph 105 | 8.76% | w/w |
| Lactose Fast Flo 316 | 8.76% | w/w |
| Ac-Di-Sol | 6.00% | w/w |
| Cab-o-sil M5P | 1.00% | w/w |
| PRUV Sodium stearyl fumarate | 0.75% | w/w |

Extra-granular Blend

| | | |
|---|---|---|
| Granules (intragranular blend above) | 84.80% | w/w |
| Avicel Ph 200 | 8.45% | w/w |
| Ac-Di-Sol | 6.00% | w/w |
| PRUV Sodium stearyl fumarate | 0.75% | w/w |

Optional Coating

| | | |
|---|---|---|
| Opadry QX 321A105082-CN Blue* | 4.00% | w/w |

*4% w/w weight gain (based on core weight)

TABLE 2
Tablet Compositions for 100 mg Tablets.

| | 100 mg Tablet | |
|---|---|---|
| Tablet Weight (mg - uncoated) | 500.0 mg | 240.0 mg |

| | | |
|---|---|---|
| | Percent by | Percent by |
| | weight | weight |
| Component | (% w/w) | (% w/w) |

Extra-granular Blend

| | | |
|---|---|---|
| SDD (Compound A:HPMC:TPGS 70/25/5) | — | 59.52% w/w |
| SDD (Compound A:HPMC:TPGS 40/55/5) | 50.00% w/w | — |
| Avicel Ph 105 | 8.18% w/w | 8.76% w/w |
| Lactose Fast Flo 316 | 8.87% w/w | 8.76% w/w |
| Ac-Di-Sol | 6.00% w/w | 6.00% w/w |
| Cab-o-sil M5P | 1.00% w/w | 1.00% w/w |
| PRUV Sodium stearyl fumarate | 0.75% w/w | 0.75% w/w |

Intra-granular Blend

| | | |
|---|---|---|
| Granules (intragranular blend above) | 74.80% w/w | 84.80% w/w |
| Avicel Ph 200 | 18.45% w/w | 8.45% w/w |
| Ac-Di-Sol | 6.00% w/w | 6.00% w/w |
| PRUV Sodium stearyl fumarate | 0.75% w/w | 0.75% w/w |

Optional Coating

| | | |
|---|---|---|
| Opadry QX 321A105082-CN Blue* | 4.00% w/w | 4.00% w/w |

*4% w/w weight gain (based on core weight)

TABLE 3
Tablet Compositions for 200 mg and 250 mg Tablets.

| | 200 mg Tablet | 250 mg Tablet |
|---|---|---|
| Tablet Weight (mg - uncoated) | 480.0 mg | 600.0 mg |

| | | |
|---|---|---|
| | Percent by weight | Percent by weight |
| Component | (% w/w) | (% w/w) |

Extra-granular Blend

| SDD (Compound A:HPMC:TPGS 70/25/5) | 59.52% | w/w | 59.52% | w/w |
|---|---|---|---|---|
| SDD (Compound A:HPMC:TPGS 40/55/5) | — | | — | |
| Avicel Ph 105 | 8.76% | w/w | 8.76% | w/w |
| Lactose Fast Flo 316 | 8.7631% | w/w | 8.76% | w/w |
| Ac-Di-Sol | 6.00% | w/w | 6.00% | w/w |
| Cab-o-sil M5P | 1.00% | w/w | 1.00% | w/w |
| PRUV Sodium stearyl fumarate | 0.75% | w/w | 0.75% | w/w |

Intra-granular Blend

| | | | | |
|---|---|---|---|---|
| Granules (intragranular blend above) | 84.80% | w/w | 84.80% | w/w |
| Avicel Ph 200 | 8.45% | w/w | 8.45% | w/w |
| Ac-Di-Sol | 6.00% | w/w | 6.00% | w/w |
| PRUV Sodium stearyl fumarate | 0.75% | w/w | 0.75% | w/w |

Optional Coating

| | | | | |
|---|---|---|---|---|
| Opadry QX 321A105082-CN Blue* | 4.00% | w/w | 4.00% | w/w |

*4% w/w weight gain (based on core weight)

### Preparation of Two Spray-Dried Dispersions

Materials used in preparation of the spray dried dispersions (SDDs) are shown below in Table 4.

TABLE 4
Materials for Spray-Dried Dispersions

| | 70/25/5 Dispersion | | | | 40/55/5 Dispersion | | | |
|---|---|---|---|---|---|---|---|---|
| Material | Relative Amount^{a} | | Tolerance | | Relative Amount | | Tolerance | |
| Compound A | 1 | kg ^{b} | ± | 1% | 1 | kg ^{b} | ± | 1% |
| Dichloromethane | 5.786 | kg | ± | 5% | 14.95 | kg | ± | 5% |
| (initial charge) | | | | | | | | |
| Methanol (initial | 1.571 | kg | ± | 5% | 5.75 | kg | ± | 5% |
| charge) | | | | | | | | |
| Vitamin E TPGS | 0.071 | kg | ± | 1% | 0.12 | kg | ± | 1% |
| Methocel E3 | 0.357 | kg | ± | 1% | 1.37 | kg | ± | 1% |
| Premium LV | | | | | | | | |
| Dichloromethane | 3.115 | kg | ± | 5% | 8.05 | kg | ± | 5% |
| (second charge) | | | | | | | | |

^{a}All values calculated relative to 1 kg Compound A, adjusted for purity. Actual amounts adjusted accordingly
^{b} Exact value, adjusted for purity

A stainless steel reactor was charged with dichloromethane and methanol under nitrogen atmosphere. The mixture was stirred with a low vortex (between 25 and 100 rpm) and the temperature was maintained at 2-8° C. (for the 70/25/5 Dispersion) or 15-25° C. (for the 40/55/5 Dispersion). Vitamin TPGS was added and the solution was stirred until an absence of solids in suspension was achieved. Methocel (i.e., HPMC) was added and the solution was stirred until an absence of solids in suspension was achieved. The temperature was adjusted to between 15-25° C (for the 70/25/5 Dispersion) and the solution was stirred for at least 30 minutes until a clear solution was achieved. Compound A was added, followed by the second charge of dichloromethane. For the 70/25/5 Dispersion, the reactor temperature was adjusted to between 2-8° C. The solution was stirred for at least 1 hour until a substantially clear solution was achieved, and then spray dried according to the parameters shown below in Table 5.

TABLE 5
Spray Dry Parameters - Compound A Solution

| | 70/25/5 Dispersion | | 40/55/5 Dispersion | |
|---|---|---|---|---|
| Parameter | Set point | Range | Set point | Range |
| Pressure (feed)^{a} | (93) | (73-113) | (96) | (76-116) |
| (bar) | | | | |
| Flow Rate (feed) | 120 | 115-125 | 120 | 115-125 |
| (kg/h) | | | | |
| Flow rate (drying) | 1400 | 1250-1550 | 1400 | 1250-1550 |
| (kg/h) | | | | |
| Temperature (in)^{b} | (85) | (75-95) | (90) | (80-100) |
| (°C) | | | | |
| Temperature (out) | 45 | 42-48 | 45 | 42-48 |
| (°C) | | | | |
| Temperature (condenser) | −7 | −10-−4 | −7 | −10-−4 |
| (°C) | | | | |

^{a}Reference values; consequence of Flow Rate (feed)
^{b}Reference values; consequence of Temperature (out)

The resulting solids were collected and transferred to a stainless steel double cone dryer and dried under vacuum until dichloromethane content 480 ppm and methanol content 2400 ppm. The drying parameters are shown below in Table 6:

TABLE 6
Post-Drying Parameters

| Parameter | Set Point | Range |
|---|---|---|
| Jacket Temperature (°C) | 40 | ≤45 |
| Stirring Speed (rpm) | 2 | N/A |
| Flow rate - Sweep | 5 | N/A |
| (Nm³/H) | | |

Once the desired solvent levels were obtained, the jacket temperature was lowered to 20° C and the product was allowed to cool to 40 °C.

### Tablet Manufacture

The intra-granular materials were blended and compacted, mixed with the extra-granular excipients, and further blended. The resulting granules were compressed into 120 mg, 240 mg, 480 mg, 500 mg, or 600 mg tablets. The resulting tablet may be film-coated.

### Example 2: Dog Studies with Amorphous and Crystalline Suspensions

Fasted female dogs were orally administered Compound A (7.5 mg/kg), as spray-dried dispersions (SDDs), pure amorphous API, or crystalline API, in aqueous suspension containing 0.5% Methocel A4M at 2 mL/kg. Animals were pretreated with famotidine at 0.5 mg/kg IV 1 hour before administration of Compound A to raise gastric pH. One group of study animals was pretreated with pentagastrin (6 μg/kg intramuscularly 1 hour before administration of crystalline Compound A as a low gastric pH comparator.

TABLE 7
Suspension Formulations Tested in Dog Pharmacokinetic Studies.

| Formulation Reference | Gastric Conditions | Type | Composition (% wt/wt) |
|---|---|---|---|
| S-A | Neutral | API | 100 Crystalline Compound A |
| S-B | Acidic | API | 100 Crystalline Compound A |
| S-C | Neutral | API | 100 Amorphous Compound A |
| S-D | Neutral | SDD | 40:60 Compound A:HPMCAS-L |
| S-E | Neutral | SDD | 40:60 Compound A:HPMC E3L V |
| S-F | Neutral | SDD | 40:55:5 Compound A:HPMC E3L V:VitE TPGS |

API = active pharmaceutical ingredient; HPMC E3LV = low viscosity E3-grade hydroxypropyl methylcellulose; HPMCAS-L = L-grade hydroxypropyl methylcellulose acetate succinate; SDD = spray-dried dispersion; VitE TPGS = D-α-tocopheryl polyethylene glycol 1000 succinate; wt = weight.

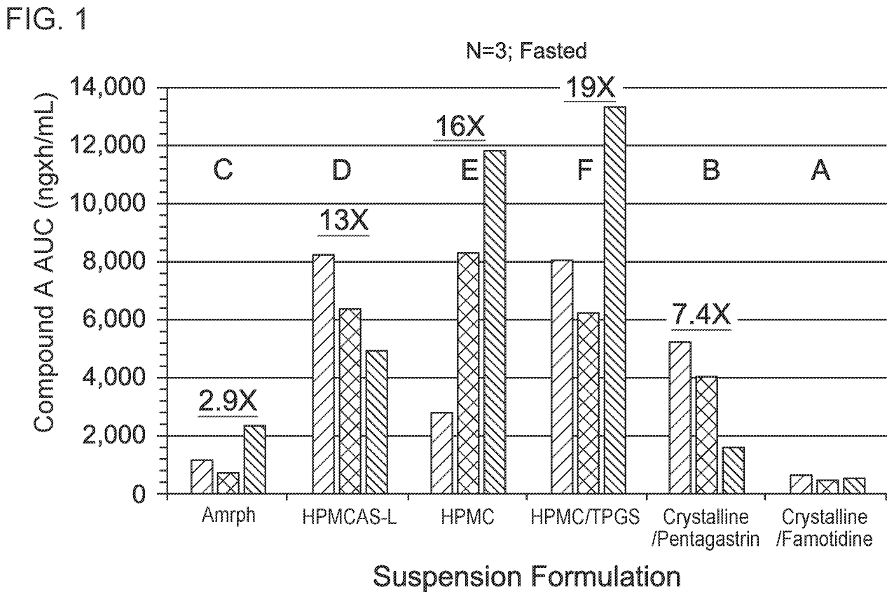

Data from the PK studies are described in Table 7 are presented in FIG. 1 and in Table 8 below.

TABLE 7

| | | Non-Normalized AUC0-24 h (ng × h/mL) | | | | | | % Fabs | |
|---|---|---|---|---|---|---|---|---|---|
| Type | Formulation | Dog 1 | Dog 2 | Dog 3 | Avg | SD | P/F Ratio | Avg | SD |
| Suspension | S-C | 1,115 | 693 | 2,349 | 1,386 | 861 | 2.9 | 6 | 4 |
| | S-D | 8,207 | 6,291 | 4,866 | 6,455 | 1,677 | 13 | 28 | 7 |
| | S-E | 2,779 | 8,291 | 11,772 | 7,614 | 4,535 | 16 | 33 | 19 |
| | S-F | 8,044 | 6,197 | 13,316 | 9,186 | 3,694 | 19 | 39 | 16 |
| | S-B | 5,158 | 4,039 | 1,539 | 3,578 | 1,853 | 7.4 | 15 | 8 |
| | S-A | 573 | 383 | 485 | 481 | 95 | — | 2.1 | 0.4 |

A large reduction in exposure was observed for crystalline material in animals with neutral gastric pH compared to that achieved under more favorable acidic gastric pH conditions. However, the amorphous polymer dispersions are able to compensate for this loss of crystalline solubility as demonstrated by the high exposure observed when Compound A is delivered by such formulations. Indeed, the exposure obtained with all polymer dispersions exceeded that afforded by crystalline free base even in the pentagastrin-pretreated dogs. Without wishing to be bound by theory, this suggests a solubility or dissolution rate limited exposure for the crystalline form. Pure, amorphous Compound A did not perform as well as the polymer dispersions. Of the polymer dispersions, hydroxypropyl methylcellulose acetate succinate (HPMC)/Vitamin E TPGS afforded the highest exposure.

### Example 3. Dog Studies with Amorphous and Crystalline Tablets

To confirm the dog PK results of the suspension formulations described above in Table 7, prototype 50 mg strength Compound A tablets were made at laboratory scale using the Compound A: low viscosity E3-grade hydroxypropyl methylcellulose (HPMC E3LV):Vitamin E TPGS (40:55:5) dispersion and crystalline drug substance (with and without an acidulant-fumaric acid) having the compositions shown in Table 8. Acidulant (fumaric acid) was added to the tablet composition to test the hypothesis of whether reducing the pH in the microenvironment surrounding the crystalline drug particles would improve bioavailability compared to crystalline drug without acidulant.

TABLE 8
Compositions of 50 mg Compound A Tablet Prototypes

| | | Formulation Reference | | | |
|---|---|---|---|---|---|
| | | T-A | T-B | T-C | T-D |
| | | Tablet Strength/Press Weight (mg/mg) | | | |
| | | 50/750 | 50/600 | 50/600 | 50/600 |
| Function | Ingredient | % of Blend | | | |
| Active | 40:55:5 Compound A:HPMC E3LV:VitE TPGS SDD | 50 | — | — | — |
| Active | Crystalline Compound A Free Base (D50 = 33 μm) | — | 25 | 25 | — |
| Active | Crystalline Compound A Free Base (D50 = 6 μm) | — | — | — | 25 |
| Acidulant | Fumaric acid | — | — | 25 | 25 |
| Filler | Microcrystalline cellulose | 21 | 35 | 22.5 | 22.5 |
| Filler | Lactose monohydrate | 21 | 35 | 22.5 | 22.5 |
| Disintegrant | Croscarmellose sodium | 6 | 3 | 3 | 3 |
| Glidant | Silicon dioxide | 1 | 1 | 1 | 1 |
| Lubricant | Sodium stearyl fumarate | 1 | 1 | 1 | 1 |

| | | | | |
|---|---|---|---|---|
| Totals: | 100 | 100 | 100 | 100 |

D₅₀ = size below which 50% of the material volume is present; HPMC E3LV = low viscosity E3-grade hydroxypropyl methylcellulose; SDD = spray-dried dispersion; VitE TPGS = D-α-tocopheryl polyethylene glycol 1000 succinate.

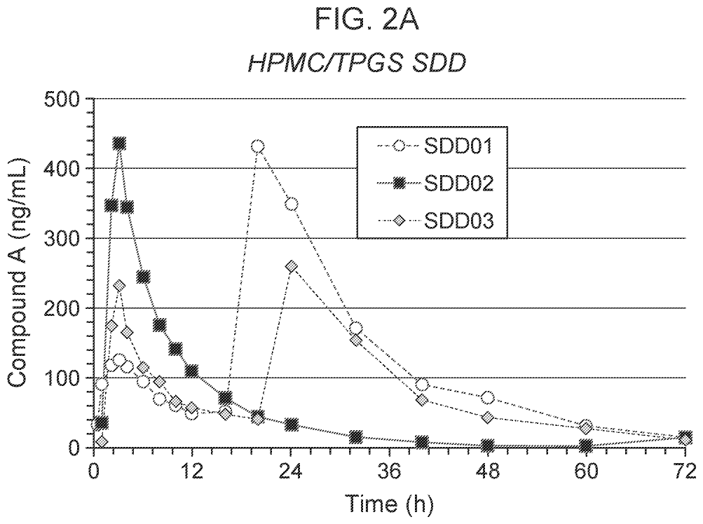

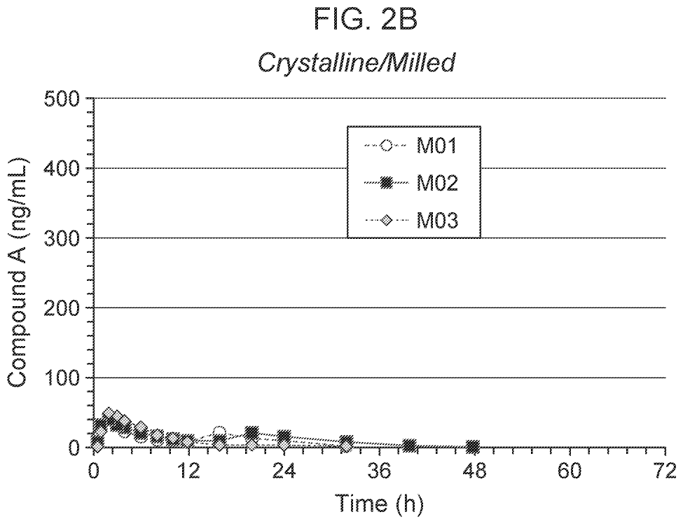

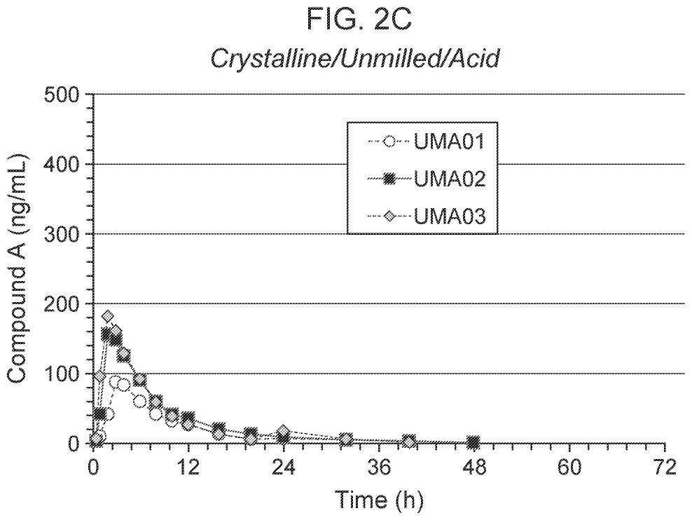

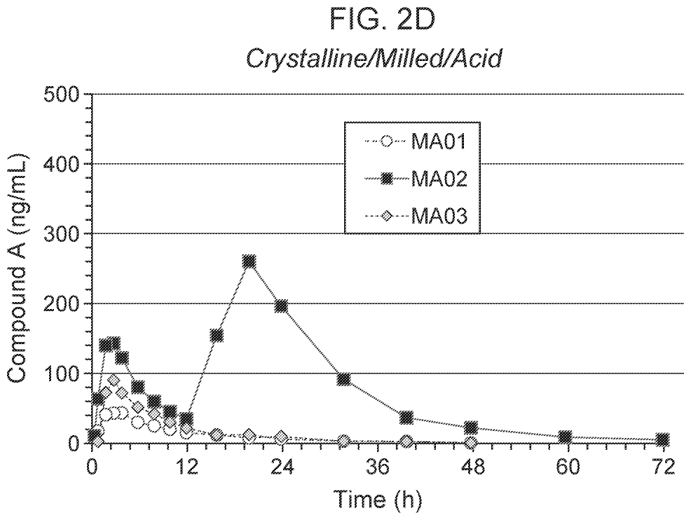

Female dogs were pretreated with famotidine at 0.5 mg/kg IV 1 hour before administration of a single 50 mg Compound A tablet. Study animals were either fasted (subsequently fed 4 hours after Compound A administration) or fed (0.5 hours before dosing) Results in fasted study animals, shown in FIGS. 2A-2D, show that exposure of size-reduced API was slightly improved by microenvironmental acidification (using fumaric acid)—see comparison of FIG. 2B vs 2D. It was also noted that increased particle size does not affect exposure in the presence of acid—see comparison of FIG. 2C vs 2D. Overall, the spray-dried dispersion in HPMC and TPGS provided the best exposure.

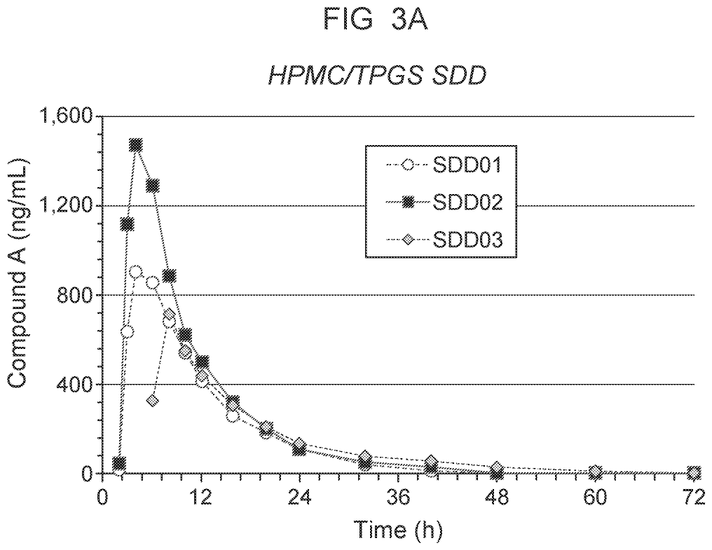

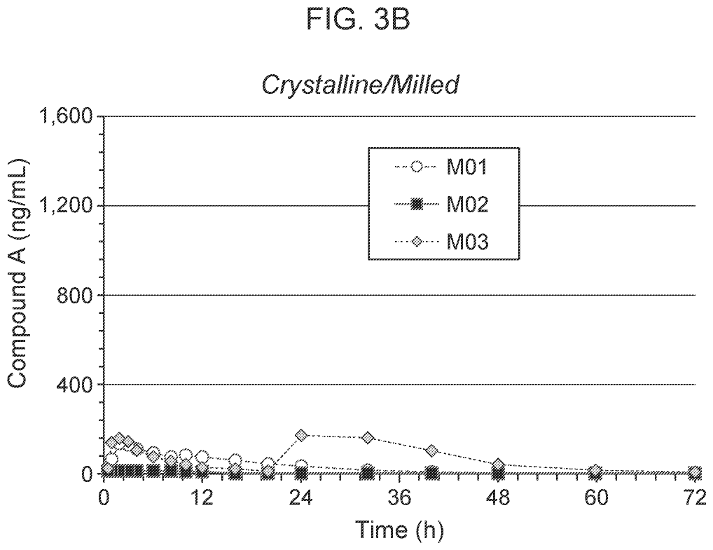

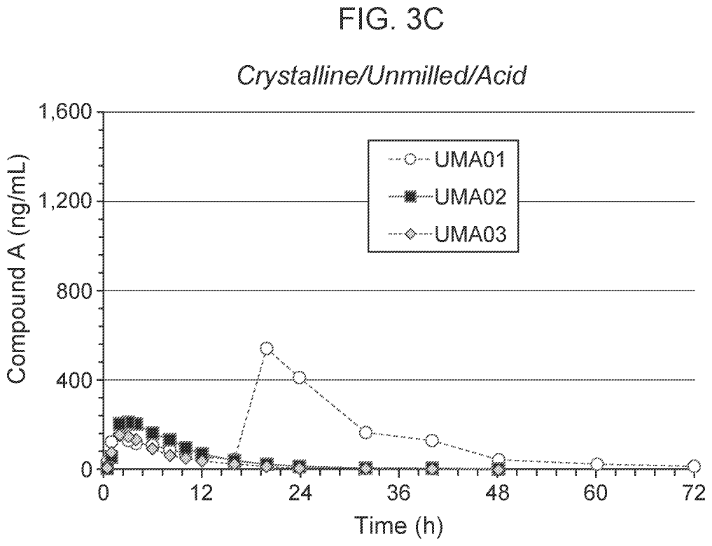

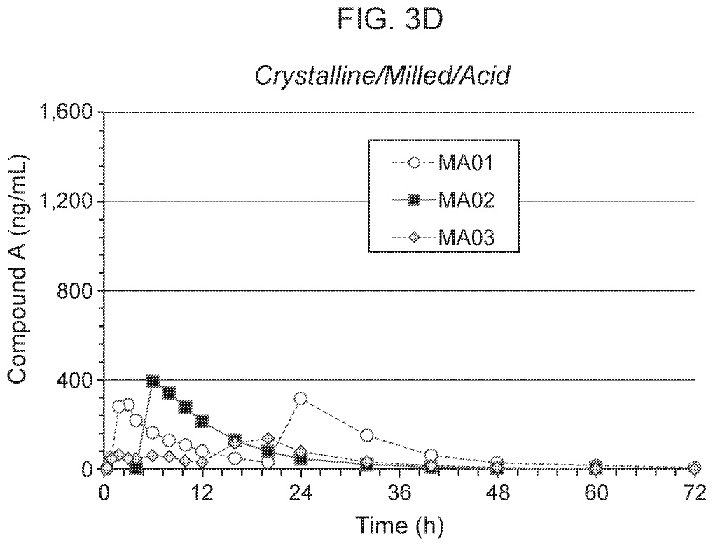

Results in fed study animals, shown in FIGS. 3A-3D, showed an approximately three-fold increase in exposure for all formulations compared to fasted subjects. As with fasted subjects, the spray-dried dispersion in HPMC and TPGS provided the best exposure.

We claim:

1. A solid oral dosage form comprising Compound A: 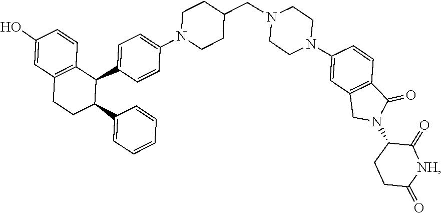 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, a polymer, and a surfactant, wherein the amount of Compound A in the solid oral dosage form is about 5 mg to about 500 mg and the solid oral dosage form is a tablet, a sachet, or a capsule.

2. The solid oral dosage form of claim 1, wherein the solid oral dosage form is a tablet.

3. A tablet comprising Compound A: 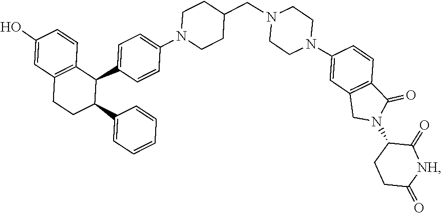 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, a polymer, and a surfactant, wherein the amount of Compound A in the solid oral dosage form is about 5 mg to about 500 mg.

4. The solid oral dosage form or the tablet of any one of claims 1-3, wherein the polymer is hydroxypropyl methylcellulose (HPMC), a hydroxypropyl methylcellulose derivative, a polyvinylpyrrolidone copolymer, a methacrylic acid copolymer, polyethylene glycol, or a polyethylene glycol derivative.

5. The solid oral dosage form or the tablet of any one of claims 1-4, wherein the polymer is hydroxypropyl methylcellulose.

6. The solid oral dosage form or the tablet of any one of claims 1-5, wherein the surfactant is selected from polyethylene glycol, a polyethylene glycol ester, glycerol esters, and mixtures thereof.

7. The solid oral dosage form or the tablet of any one of claims 1-6, wherein the surfactant is a vitamin E ester of polyethylene glycol.

8. The solid oral dosage form or the tablet of any one of claims 1-7, wherein the surfactant is D-α-tocopheryl polyethylene glycol succinate.

9. The solid oral dosage form or the tablet of any one of claims 1-8, wherein the surfactant is D-α-tocopheryl polyethylene glycol 1000 succinate.

10. The solid oral dosage form or the tablet of claim 1, wherein the polymer is hydroxypropyl methylcellulose, and the surfactant is D-α-tocopheryl polyethylene glycol succinate.

11. The solid oral dosage form or the tablet of any one of claims 1-10, further comprising one or more excipients selected from fillers, disintegrants, glidants, and lubricants.

12. The solid oral dosage form or the tablet of claim 11, wherein the filler is microcrystalline cellulose, silicified microcrystalline cellulose, lactose monohydrate, mannitol, sorbitol, xylitol, hydroxypropyl methylcellulose, hydroxypropyl cellulose, pullulan, fast-dissolving carbohydrates, or a combination thereof.

13. The solid oral dosage form or the tablet of claim 11, wherein the disintegrant is sodium starch glycolate, sodium carboxymethyl cellulose, calcium carboxymethyl cellulose, croscarmellose sodium, crospovidone, chitosan, agar, alginic acid, calcium alginate, methyl cellulose, microcrystalline cellulose, powdered cellulose, lower alkyl substituted hydroxypropyl cellulose, hydroxylpropyl starch, low-substituted hydroxypropylcellulose, polacrilin potassium, starch, pregelatinized starch, sodium alginate, polacrilin potassium, povidone, or a combination thereof.

14. The solid oral dosage form or the tablet of claim 11, wherein the glidant is silicon dioxide, colloidal silicon dioxide, calcium silicate, magnesium silicate, magnesium trisilicate, talc, starch, or a combination thereof.

15. The solid oral dosage form or the tablet of claim 11, wherein the lubricant is magnesium stearate, calcium stearate, glyceryl monostearate, glyceryl behenate, glyceryl palmitostearate, hexagonal boron nitride, hydrogenated vegetable oil, light mineral oil, mineral oil, polyethylene glycol, poloxamer, sodium benzoate, sodium lauryl sulfate, sodium stearyl fumarate, stearic acid, talc, zinc stearate, or any combination thereof.

16. The solid oral dosage form or the tablet of any one of claims 1-3 and 10-15, comprising:
about 15% w/w to about 50% w/w of Compound A;
about 10% w/w to about 40% w/w of hydroxypropyl methylcellulose;
about 0.5% w/w to about 5% w/w of D-α-tocopheryl polyethylene glycol succinate;
about 10% w/w to about 40% w/w of microcrystalline cellulose;
about 5% w/w to about 15% w/w of lactose monohydrate;
about 5% w/w to about 15% w/w of croscarmellose sodium;
about 0% w/w to about 5% w/w of silicon dioxide; and
about 0% w/w to about 2% w/w of sodium stearyl fumarate.

17. The solid oral dosage form or the tablet of any one of claims 1-3 and 10-15, comprising:
15% to 50% w/w of Compound A;
10% to 40% w/w of hydroxypropyl methylcellulose;
0.5% to 5% w/w of D-α-tocopheryl polyethylene glycol succinate;
10% to 40% w/w of microcrystalline cellulose;
5% to 15% w/w of lactose monohydrate;
5% to 15% w/w of croscarmellose sodium;
0% to 5% w/w of silicon dioxide; and
0% to 2% w/w of sodium stearyl fumarate.

18. The solid oral dosage form or the tablet of any one of claims 1-3 and 10-15, comprising:
about 42% w/w of Compound A;
about 15% w/w of hydroxypropyl methylcellulose;
about 3% w/w of D-α-tocopheryl polyethylene glycol succinate;
about 17% w/w of microcrystalline cellulose;
about 8.8% w/w of lactose monohydrate;
about 12% w/w of croscarmellose sodium;
about 1% w/w of silicon dioxide; and
about 1.5% w/w of sodium stearyl fumarate.

19. The solid oral dosage form or the tablet of any one of claims 1-3 and 10-15, comprising:
about 20% w/w of Compound A;
about 28% w/w of hydroxypropyl methylcellulose;
about 2.5% w/w of D-α-tocopheryl polyethylene glycol succinate;
about 27% w/w of microcrystalline cellulose;
about 9% w/w of lactose monohydrate;
about 12% w/w of croscarmellose sodium;
about 1% w/w of silicon dioxide; and
about 1.5% w/w of sodium stearyl fumarate.

20. The solid oral dosage form or the tablet of any one of claims 1-3 and 10-15, comprising an intra-granular portion and an extra-granular portion.

21. The solid oral dosage form or the tablet of claim 20, wherein the intra-granular portion comprises:
about 15% to about 50% w/w of Compound A;
about 10% to about 40% w/w of hydroxypropyl methylcellulose;
about 0.5% to about 5% w/w of D-α-tocopheryl polyethylene glycol succinate;
about 5% to about 10% w/w of microcrystalline cellulose;
about 5% to about 10% w/w of lactose monohydrate;
about 1% to about 10% w/w of croscarmellose sodium;
about 0% to about 5% w/w of silicon dioxide; and
about 0% to about 2% w/w of sodium stearyl fumarate,
and wherein the extra-granular portion comprises:
about 5% to about 25% w/w of microcrystalline cellulose;
about 0% to about 10% w/w of croscarmellose sodium; and
about 0% to about 2% w/w of sodium stearyl fumarate;
wherein weight percentages are relative to the total uncoated tablet weight.

22. The solid oral dosage form or the tablet of claim 20, wherein the intra-granular portion comprises:
about 42% w/w of Compound A;
about 14% w/w hydroxypropyl methylcellulose;
about 3% w/w d-α-tocopheryl polyethylene glycol succinate;
about 9% w/w microcrystalline cellulose;
about 9% w/w lactose monohydrate;
about 6% w/w croscarmellose sodium;
about 1% w/w silicon dioxide; and
about 0.75% w/w sodium stearyl fumarate;
and wherein the extra-granular portion comprises:
about 8% w/w microcrystalline cellulose;
about 6% w/w croscarmellose sodium; and
about 0.75% w/w sodium stearyl fumarate;
wherein weight percentages are relative to the total uncoated tablet weight.

23. The solid oral dosage form or the tablet of claim 20, wherein the intra-granular portion comprises:
about 20% w/w of Compound A;
about 28% w/w of hydroxypropyl methylcellulose;
about 2.5% w/w of D-α-tocopheryl polyethylene glycol succinate;
about 8% w/w of microcrystalline cellulose;
about 9% w/w of lactose monohydrate;
about 6% w/w of croscarmellose sodium;
about 1% w/w of silicon dioxide; and
about 0.75% w/w of sodium stearyl fumarate,
and wherein the extra-granular portion comprises:
about 18% w/w of microcrystalline cellulose;
about 6% w/w of croscarmellose sodium; and
about 0.75% w/w of sodium stearyl fumarate;
wherein weight percentages are relative to the total uncoated tablet weight.

24. The solid oral dosage form or the tablet of any one of claims 1-23, wherein the tablet is film coated.

25. The solid oral dosage form or the tablet of any one of claims 1-24, wherein the amount of Compound A in the solid oral dosage form is about 5 mg, about 10 mg, about 15 mg, about 20 mg, about 25 mg, about 30 mg, about 50 mg, about 75 mg, about 100 mg, about 125 mg, about 150 mg, about 200 mg, about 250 mg, or about 300 mg.

26. The solid oral dosage form or the tablet of any one of claims 1-25, the amount of Compound A in solid oral dosage form is about 200 mg.

27. The solid oral dosage form or the tablet of any one of claims 1-25, wherein the amount of Compound A in solid oral dosage form is about 100 mg.

28. The solid oral dosage form or the tablet of any one of claims 1-25, wherein the amount of Compound A in the solid oral dosage form is about 250 mg.

29. The solid oral dosage form or the tablet of any one of claims 1-25, wherein the amount of Compound A in the solid oral dosage form is about 50 mg.

30. A mixture comprising Compound A: 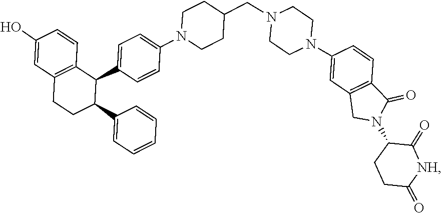 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, a polymer, and a surfactant.

31. The mixture of claim 30, wherein the polymer is hydroxypropyl methylcellulose (HPMC), a hydroxypropyl methylcellulose derivative, a polyvinylpyrrolidone copolymer, a methacrylic acid copolymer, polyethylene glycol, or a polyethylene glycol derivative.

32. The mixture form of claim 30 or 31, wherein the polymer is hydroxypropyl methylcellulose.

33. The mixture form of any one of claims 30-32, wherein the surfactant is selected from polyethylene glycol, a polyethylene glycol ester, glycerol esters, and mixtures thereof.

34. The mixture of any one of claims 30-33, wherein the surfactant is a vitamin E ester of polyethylene glycol.

35. The mixture of any one of claims 30-34, wherein the surfactant is D-α-tocopheryl polyethylene glycol succinate.

36. The mixture of any one of claims 30-35, wherein the surfactant is D-α-tocopheryl polyethylene glycol 1000 succinate.

37. The mixture of claim 30, the polymer is hydroxypropyl methylcellulose, and the surfactant is D-α-tocopheryl polyethylene glycol succinate.

38. The mixture of any one of claims 30-37, comprising:
about 20% (w/w) to about 60% (w/w) of Compound A,
about 35% (w/w) to about 75% (w/w) of the polymer, and
about 1% (w/w) to about 10% (w/w) of a surfactant.

39. The mixture of any one of claims 30-37, comprising:
about 40% (w/w) of Compound A,
about 55% (w/w) of the polymer, and
about 5% (w/w) of the surfactant.

40. The mixture of any one of claims 30-37, comprising:
about 50% to about 90% (w/w) of Compound A,
about 10% to about 40% (w/w) of the polymer, and
about 1% to about 10% (w/w) of the surfactant.

41. The mixture of any one of claims 30-37, comprising:
about 70% (w/w) Compound A,
about 25% (w/w) of the polymer, and
about 5% (w/w) of the surfactant.

42. The mixture of claim 38, comprising:
about 20% (w/w) to about 60% (w/w) of Compound A,
about 35% (w/w) to about 75% (w/w) of hydroxypropyl methylcellulose, and
about 1% (w/w) to about 10% (w/w) of D-α-tocopheryl polyethylene glycol succinate.

43. The mixture of claim 39, comprising:
about 40% (w/w) of Compound A,
about 55% (w/w) of hydroxypropyl methylcellulose, and
about 5% (w/w) of D-α-tocopheryl polyethylene glycol succinate.

44. The mixture of claim 40, comprising:
about 50% to about 90% (w/w) of Compound A,
about 10% to about 40% (w/w) of hydroxypropyl methylcellulose, and
about 1% to about 10% (w/w) of D-α-tocopheryl polyethylene glycol succinate.

45. The mixture of claim 41, comprising:
about 70% (w/w) Compound A,
about 25% (w/w) hydroxypropyl methylcellulose, and
about 5% (w/w) D-α-tocopheryl polyethylene glycol succinate.

46. The mixture of any one of claims 30-45, wherein the mixture is a dispersion.

47. The mixture of claim 46, wherein the dispersion is a solid dispersion.

48. The mixture of claim 46 or 47, wherein the dispersion is produced by spray drying, lyophilization, hot melt extrusion, milling, solvent evaporation, supercritical fluid processing, or high-shear mixing.

49. The mixture of any one of claims 46-48, wherein in the wherein the dispersion is produced by spray drying.

50. A spray dried dispersion comprising Compound A: 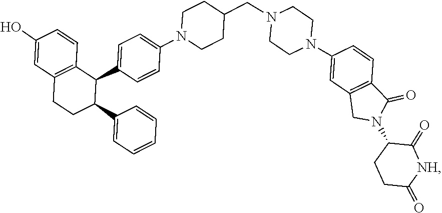 [SMILES: O=C1CC[C@H](N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]
or a pharmaceutically acceptable salt thereof, a polymer, and a surfactant.

51. The spray dried dispersion of claim 50, wherein the polymer is hydroxypropyl methylcellulose (HPMC), a hydroxypropyl methylcellulose derivative, a polyvinylpyrrolidone copolymer, a methacrylic acid copolymer, polyethylene glycol, or a polyethylene glycol derivative.

52. The spray dried dispersion of claim 50 or 51, wherein the polymer is hydroxypropyl methylcellulose.

53. The spray dried dispersion of any one of claims 50-52, wherein the surfactant is selected from polyethylene glycol, a polyethylene glycol ester, glycerol esters, and mixtures thereof.

54. The spray dried dispersion of any one of claims 50-53, wherein the surfactant is a vitamin E ester of polyethylene glycol.

55. The spray dried dispersion of any one of claims 50-54, wherein the surfactant is D-α-tocopheryl polyethylene glycol succinate.

56. The spray dried dispersion of any one of claims 50-55, wherein the surfactant is D-α-tocopheryl polyethylene glycol 1000 succinate.

57. The spray dried dispersion of claim 50, the polymer is hydroxypropyl methylcellulose, and the surfactant is D-α-tocopheryl polyethylene glycol succinate.

58. The spray dried dispersion of claim 50, comprising:
about 20% (w/w) to about 60% (w/w) of Compound A,
about 35% (w/w) to about 75% (w/w) of the polymer, and
about 1% (w/w) to about 10% (w/w) of the surfactant.

59. The spray dried dispersion of claim 50, comprising:
about 40% (w/w) of Compound A,
about 55% (w/w) of the polymer, and
about 5% (w/w) of the surfactant.

60. The spray dried dispersion of claim 50, comprising:
about 50% to about 90% (w/w) of Compound A,
about 10% to about 40% (w/w) of the polymer, and
about 1% to about 10% (w/w) of the surfactant.

61. The spray dried dispersion of claim 50, comprising:
about 70% (w/w) Compound A,
about 25% (w/w) of the polymer, and
about 5% (w/w) of a surfactant.

62. The spray dried dispersion of claim 51, comprising:
about 20% (w/w) to about 60% (w/w) of Compound A,
about 35% (w/w) to about 75% (w/w) of hydroxypropyl methylcellulose, and
about 1% (w/w) to about 10% (w/w) of D-α-tocopheryl polyethylene glycol succinate.

63. The spray dried dispersion of claim 59, comprising:
about 40% (w/w) of Compound A,
about 55% (w/w) of hydroxypropyl methylcellulose, and
about 5% (w/w) of D-α-tocopheryl polyethylene glycol succinate.

64. The spray dried dispersion of claim 60, comprising:
about 50% to about 90% (w/w) of Compound A,
about 10% to about 40% (w/w) of hydroxypropyl methylcellulose, and
about 1% to about 10% (w/w) of D-α-tocopheryl polyethylene glycol succinate.

65. The spray dried dispersion of claim 61, comprising:
about 70% (w/w) Compound A,
about 25% (w/w) hydroxypropyl methylcellulose, and
about 5% (w/w) D-α-tocopheryl polyethylene glycol succinate.

66. A method of making the spray dried dispersion of any one of claims 50-65, comprising
Dissolving Compound A, the polymer, and the surfactant in a solvent to afford a solution comprising Compound A;
Introducing the solution comprising Compound A into a spray dryer;
Spraying the solution comprising Compound A from the spray dryer to form a spray dried dispersion of Compound A; and
Optionally, removing the residual solvent from the spray dried dispersion of Compound A.

67. The method of claim 66, wherein the solvent is a mixture of dichloromethane and methanol.

68. The method of claim 66, wherein the solvent is a mixture of about 90:10 (w/w) to about 70:30 (w/w) dichloromethane:methanol.

69. The method of claim 66, wherein the solvent is a mixture of about 80:20 (w/w) dichloromethane:methanol.

70. The method of claim 66, wherein the solvent is a mixture of about 85:15 (w/w) dichloromethane:methanol.

71. The method of any one of claims 66-70, wherein removing residual solvent comprises drying.

72. The method of claim 71, wherein drying comprises agitated conical drying.
